SEQUENCE LISTING (12 sequences)

SEQ ID NO: 1 - DNA, 5425 bp, Homo sapiens
  CDS (181)..(5079)
ggctccgttccatccttctgcacagggtatcgcctctctccgtttggtacatcccctcctcccccacgcccggactggggtggtagacgccgcctccgctcatcgcccctccccatcggtttccgcgcgaaaagccggggcgcctgcgctgccgccgccgcgtctgctgaagcctccgagatgccggcgcgtaccgccccagcccgggtgcccacactggccgtcccggccatctcgctgcccgacgatgtccgcaggcggctcaaagatttggaaagagacagcttaacagaaaaggaatgtgtgaaggagaaattgaatctcttgcacgaatttctgcaaacagaaataaagaatcagttatgtgacttggaaaccaaattacgtaaagaagaattatccgaggagggctacctggctaaagtcaaatcccttttaaataaagatttgtccttggagaacggtgctcatgcttacaaccgggaagtgaatggacgtctagaaaacgggaaccaagcaagaagtgaagcccgtagagtgggaatggcagatgccaacagcccccccaaacccctttccaaacctcgcacgcccaggaggagcaagtccgatggagaggctaagcgttcaagagaccctcctgcctcagcctcccaagtaactgggattagagctgaaccttcacctagccccaggattacaaggaaaagcaccaggcaaaccaccatcacatctcattttgcaaagggccctgccaaacggaaacctcaggaagagtctgaaagagccaaatcggatgagtccatcaaggaagaagacaaagaccaggatgagaagagacgtagagttacatccagagaacgagttgctagaccgcttcctgcagaagaacctgaaagagcaaaatcaggaacgcgcactgaaaaggaagaagaaagagatgaaaaagaagaaaagagactccgaagtcaaaccaaagaaccaacacccaaacagaaactgaaggaggagccggacagagaagccagggcaggcgtgcaggctgacgaggacgaagatggagacgagaaagatgagaagaagcacagaagtcaacccaaagatctagctgccaaacggaggcccgaagaaaaagaacctgaaaaagtaaatccacagatttctgatgaaaaagacgaggatgaaaaggaggagaagagacgcaaaacgacccccaaagaaccaacggagaaaaaaatggctcgcgccaaaacagtcatgaactccaagacccaccctcccaagtgcattcagtgcgggcagtacctggacgaccctgacctcaaatatgggcagcacccaccagacgcggtggatgagccacagatgctgacaaatgagaagctgtccatctttgatgccaacgagtctggctttgagagttatgaggcgcttccccagcacaaactgacctgcttcagtgtgtactgtaagcacggtcacctgtgtcccatcgacaccggcctcatcgagaagaatatcgaactcttcttttctggttcagcaaaaccaatctatgatgatgacccatctcttgaaggtggtgttaatggcaaaaatcttggccccataaatgaatggtggatcactggctttgatggaggtgaaaaggccctcatcggcttcagcacctcatttgccgaatacattctgatggatcccagtcccgagtatgcgcccatatttgggctgatgcaggagaagatctacatcagcaagattgtggtggagttcctgcagagcaattccgactcgacctatgaggacctgatcaacaagatcgagaccacggttcctccttctggcctcaacttgaaccgcttcacagaggactccctcctgcgacacgcgcagtttgtggtggagcaggtggagagttatgacgaggccggggacagtgatgagcagcccatcttcctgacaccctgcatgcgggacctgatcaagctggctggggtcacgctgggacagaggcgagcccaggcgaggcggcagaccatcaggcattctaccagggagaaggacaggggacccacgaaagccaccaccaccaagctggtctaccagatcttcgatactttcttcgcagagcaaattgaaaaggatgacagagaagacaaggagaacgcctttaagcgccggcgatgtggcgtctgtgaggtgtgtcagcagcctgagtgtgggaaatgtaaagcctgcaaggacatggttaaatttggtggcagtggacggagcaagcaggcttgccaagagcggaggtgtcccaatatggccatgaaggaggcagatgacgatgaggaagtcgatgataacatcccagagatgccgtcacccaaaaaaatgcaccaggggaagaagaagaaacagaacaagaatcgcatctcttgggtcggagaagccgtcaagactgatgggaagaagagttactataagaaggtgtgcattgatgcggaaaccctggaagtgggggactgtgtctctgttattccagatgattcctcaaaaccgctgtatctagcaagggtcacggcgctgtgggaggacagcagcaacgggcagatgtttcacgcccactggttctgcgctgggacagacacagtcctcggggccacgtcggaccctctggagctgttcttggtggatgaatgtgaggacatgcagctttcatatatccacagcaaagtgaaagtcatctacaaagccccctccgaaaactgggccatggagggaggcatggatcccgagtccctgctggagggggacgacgggaagacctacttctaccagctgtggtatgatcaagactacgcgagattcgagtcccctccaaaaacccagccaacagaggacaacaagttcaaattctgtgtgagctgtgcccgtctggctgagatgaggcaaaaagaaatccccagggtcctggagcagctcgaggacctggatagccgggtcctctactactcagccaccaagaacggcatcctgtaccgagttggtgatggtgtgtacctgccccctgaggccttcacgttcaacatcaagctgtccagtcccgtgaaacgcccacggaaggagcccgtggatgaggacctgtacccagagcactaccggaaatactccgactacatcaaaggcagcaacctggatgcccctgagccctaccgaattggccggatcaaagagatcttctgtcccaagaagagcaacggcaggcccaatgagactgacatcaaaatccgggtcaacaagttctacaggcctgagaacacccacaagtccactccagcgagctaccacgcagacatcaacctgctctactggagcgacgaggaggccgtggtggacttcaaggctgtgcagggccgctgcaccgtggagtatggggaggacctgcccgagtgcgtccaggtgtactccatgggcggccccaaccgcttctacttcctcgaggcctataatgcaaagagcaaaagctttgaagatcctcccaaccatgcccgtagccctggaaacaaagggaagggcaagggaaaagggaagggcaagcccaagtcccaagcctgtgagccgagcgagccagagatagagatcaagctgcccaagctgcggaccctggatgtgttttctggctgcggggggttgtcggagggattccaccaagcaggcatctctgacacgctgtgggccatcgagatgtgggaccctgcggcccaggcgttccggctgaacaaccccggctccacagtgttcacagaggactgcaacatcctgctgaagctggtcatggctggggagaccaccaactcccgcggccagcggctgccccagaagggagacgtggagatgctgtgcggcgggccgccctgccagggcttcagcggcatgaaccgcttcaattcgcgcacctactccaagttcaaaaactctctggtggtttccttcctcagctactgcgactactaccggccccggttcttcctcctggagaatgtcaggaactttgtctccttcaagcgctccatggtcctgaagctcaccctccgctgcctggtccgcatgggctatcagtgcaccttcggcgtgctgcaggccggtcagtacggcgtggcccagactaggaggcgggccatcatcctggccgcggcccctggagagaagctccctctgttcccggagccactgcacgtgtttgctccccgggcctgccagctgagcgtggtggtggatgacaagaagtttgtgagcaacataaccaggttgagctcgggtcctttccggaccatcacggtgcgagacacgatgtccgacctgccggaggtgcggaatggagcctcggcactggagatctcctacaacggggagcctcagtcctggttccagaggcagctccggggcgcacagtaccagcccatcctcagggaccacatctgtaaggacatgagtgcattggtggctgcccgcatgcggcacatccccttggccccagggtcagactggcgcgatctgcccaacatcgaggtgcggctctcagacggcaccatggccaggaagctgcggtatacccaccatgacaggaagaacggccgcagcagctctggggccctccgtggggtctgctcctgcgtggaagccggcaaagcctgcgaccccgcagccaggcagttcaacaccctcatcccctggtgcctgccccacaccgggaaccggcacaaccactgggctggcctctatggaaggctcgagtgggacggcttcttcagcacaaccgtcaccaaccccgagcccatgggcaagcagggccgcgtgctccacccagagcagcaccgtgtggtgagcgtgcgggagtgtgcccgctcccagggcttccctgacacctaccggctcttcggcaacatcctggacaagcaccggcaggtgggcaatgccgtgccaccgcccctggccaaagccattggcttggagatcaagctttgtatgttggccaaagcccgagagagtgcctcagctaaaataaaggaggaggaagctgctaaggactagttctgccctcccgtcacccctgtttctggcaccaggaatccccaacatgcactgatgttgtgtttttaacatgtcaatctgtccgttcacatgtgtggtacatggtgtttgtggccttggctgacatgaagctgttgtgtgaggttcgcttatcaactaatgatttagtgatcaaattgtgcagtactttgtgcattctggattttaaaagttttttattatgcattatatcaaatctaccactgtatgagtggaaattaagactttatgtagtttttatatgttgtaatatttcttcaaataaatctctcctataaaccaaaaaaaaaaaaaaaaaaaaaaaaaa

SEQ ID NO: 2 - protein, 1632 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKRSRDPPASASQVTGIRAEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 3 - DNA, 5367 bp, Mus musculus
  CDS (193)..(5052)
tagccaggaggtgtgggtgcctccgttgcgcgcatgcgcactcccttcgggcatagcatggtcttcccccactctcttgccctgtgtggtacatgctgcttccgcttgcgccgccccctcccaattggtttccgcgcgcgcgaaaaagccggggtctcgttcagagctgttctgtcgtctgcaacctgcaagatgccagcgcgaacagctccagcccgagtgcctgcgcttgcctccccggcaggctcgctcccggaccatgtccgcaggcggctcaaagacttggaaagagatggcttaacagaaaaggagtgtgtgagggagaaattaaacttactgcatgaattcctgcaaacagaaataaaaagccagttgtgtgacttggaaaccaaattacataaagaggaattatctgaggaaggctacctggctaaagtcaagtccctcttaaataaggatttgtccttggagaacggaacacacactctcactcaaaaagccaacggttgtcccgccaacgggagccggccaacctggagagcagaaatggcagactcaaatagatccccaagatccaggcccaagcctcggggacccaggagaagcaagtcggacagtgacaccctttttgaaacttcacctagttccgtggctacgaggagaaccaccaggcagaccaccatcacggctcacttcacgaagggccccactaaacggaaacccaaggaagagtcggaagaggggaactcggctgagtcggctgcagaggagagagaccaggataagaaacgcagagttgtagacacagagagtggtgctgcagctgctgtggagaaactggaagaggtaacagcgggaacccagctgggtccggaagagccatgtgaacaggaagatgacaacaggagtcttcgacgtcacaccagagagctatcattgaggcggaaatcaaaggaggatccagacagagaagcaagaccggaaactcacttggacgaggacgaggacggaaaaaaggataaaagaagttccagacccaggagccagcccagagatccagctgccaaacggagacccaaggaagcagagccagagcaggtagctccagagactcccgaggacagagacgaggatgagagggaggagaagagacgaaaaacgacacgtaaaaaactggagtcacacaccgttcccgttcagagcagatcggagagaaaagccgctcaaagcaaaagtgtgatcccgaagatcaactcaccaaagtgccccgagtgtggccagcacctagacgaccctaacctgaagtaccagcagcaccctgaggatgctgtggatgaaccccagatgttgaccagtgagaaactgtccatctacgactccacctcgacctggtttgatacttatgaagattctcccatgcataggttcacttccttcagtgtgtactgcagtcgcgggcacctgtgtcctgtcgacaccggtctcattgagaagaatgtagagctctacttttctgggtgtgccaaagcaattcatgacgagaatccatctatggaaggtggtattaatggcaaaaacctcgggccaatcaatcagtggtggctcagtggctttgatggtggcgagaaggtgctcattggcttctccactgcatttgctgaatacattttgatggagcccagcaaagagtatgagccaatatttgggctgatgcaggagaaaatttacatcagcaagattgttgttgagttcctgcaaaacaatcctgatgctgtatatgaagacctgatcaataagattgagaccactgttcctccttctaccattaatgtgaaccggttcacagaggactccctcttacgccacgcccagtttgtagtgagccaggtagagagttacgacgaagccaaggacgatgatgagacccccatcttcttgtctccctgtatgagagccctgatccatttggctggtgtctccctgggacagaggcgagcaacaaggcgcgtcatgggtgctaccaaggagaaggacaaagcacccacgaaagccaccaccaccaagctggtctatcagatctttgacactttcttctcagagcagattgagaagtatgataaggaggacaaggagaatgccatgaagcgccgccgctgtggtgtctgtgaggtctgtcagcagcctgagtgtgggaagtgcaaggcgtgcaaagatatggtgaagtttggtggcactggacggagtaagcaggcttgcctcaagaggaggtgtcctaacttggcggtgaaggaggcagacgacgatgaagaggctgatgatgatgtgtcagagatgccatcacccaaaaagctgcatcaggggaagaagaagaagcagaacaaggaccgcatctcctggcttgggcagcctatgaagattgaagagaatagaacttactatcagaaggtgagcatcgatgaggagatgctagaggtgggcgactgcgtctcggtcattccagatgattcctccaaaccactctatctagccagggtcacagctctgtgggaagacaaaaatggtcagatgatgttccatgcgcactggttctgcgctgggacagacacagtcctgggagccacctccgaccccctggaactgttcctggtgggcgagtgcgaaaacatgcagctttcctacatccacagcaaggtcaaggtcatctacaaagccccttctgaaaactgggccatggagggaggcacagaccctgagaccacactgcctggggctgaggatggcaagacttacttcttccagctctggtacaaccaggagtacgcaaggtttgaatccccacccaagacccagccgaccgaggacaacaagcacaagttctgcctatcttgtatccggctggctgagctgagacaaaaagaaatgcccaaggtcctggaacaaattgaggaggtggatggccgggtctactgcagttccatcaccaagaatggtgttgtctaccgactgggtgacagtgtgtaccttcctcccgaggcctttactttcaacatcaaagtggctagccccgtgaaacgcccaaagaaggatcctgtgaacgagaccctgtaccctgagcactaccgcaagtattctgactacatcaaggggagcaacctggatgctccagagccctatcgcatcggtcggataaaagagatccactgtggcaagaagaaaggcaaggtcaacgaggcagacatcaagctgaggctctacaagttctacaggcctgagaatacccacaggtcctacaacggatcctatcacactgacatcaacatgctttactggagcgacgaggaagctgtggtgaacttcagcgacgtgcagggccgctgtaccgtggagtacggggaagacctacttgagagcatccaggattattcacaagggggccctgaccgcttctacttcctcgaggcctacaattcaaagaccaagaactttgaagacccaccaaaccatgcccgcagccctgggaacaaagggaaagggaaggggaaagggaaggggaaggggaagcatcaggtgtcagagcccaaagagcctgaggcagccatcaaactgcccaagctccggaccctggatgtgttttccggctgtggagggttatcggaaggattccaccaagcaggcatctcggaaacgctgtgggccatcgagatgtgggacccggcagcccaggcatttcggctgaacaaccccggcaccacagtgttcacagaggactgcaacgtgcttcttaagctggtcatggctggggaggtgaccaactctctgggccaaaggctgccacagaagggcgatgtggagatgctgtgtggtgggccaccctgccagggcttcagtggcatgaaccgcttcaactcccgcacttactccaagttcaaaaactccctagtggtctccttcctcagctactgtgactactaccggcctcggttcttccttctggagaacgtcaggaacttcgtgtcctacagacgctccatggtgctgaagctcacactgcgctgcctggtccgcatgggctaccagtgcacctttggtgtgctccaggctggacagtatggcgtggcccagacacgaaggagggccatcatcttggctgcagccccaggagaaaagctgcctctgttcccagagcctctgcatgtgtttgcgccccgtgcctgccagctgagcgttgtggtggatgacaagaagtttgttagcaacataacgaggctgagctcggggcccttccgaaccatcaccgtgcgagacaccatgtctgacctccccgagatccagaatggagcctcgaattctgagatcccctacaatggagagccactgtcctggttccagaggcagctgcgaggatcacactaccagcccatcctcagggaccatatctgcaaggacatgagcccactggtggctgcccgcatgcggcacatcccactgttcccaggatcagattggcgtgacctgcccaacatacaggtgcggctgggagatggcgtcatagcccataagctacagtacacctttcatgatgtgaaaaatggctacagcagcaccggtgccctgcgtggagtctgttcctgtgcagaaggcaaggcctgcgaccctgagtccaggcaattcagcaccctcatcccctggtgcctgccgcacactgggaaccggcacaaccactgggctggcctctacgggcgtctggagtgggatggcttcttcagcaccactgtcaccaaccctgagcccatgggcaagcagggtcgggtgctccacccggagcagcaccgggtcgtgagtgttcgggaatgtgcccgctcccagggctttccagatagctaccggttcttcggcaacatcctggacagacaccggcaggtgggtaatgctgtgccaccacccctggccaaagccattggcctggagattaagctctgcctgctgtccagtgctcgggagagcgcatcagctgcagttaaagcaaaagaggaggctgctaccaaggactagtgctctcacccagagccccacgtgcactgatgtttttaaccctttgagccccatcatttgaagtcttgtgctcagtgtctgtggccatggctgacactaagctgtttgtatgaggtttgttttgtgaccaagctgtgtagtactttgtgcattctgaattttaaggtttttttttttgtttggtttggtttggtttggtttttttcttatcctgtattctatcagatctgccactgtgcaggtggcaagtgagacttgatgtagttttatatgttgtaatatttcttcaaaataaagcgcttctgtcaagcaccc

SEQ ID NO: 4 - protein, 1619 aa, Mus musculus
MPARTAPARVPALASPAGSLPDHVRRRLKDLERDGLTEKECVREKLNLLHEFLQTEIKSQLCDLETKLHKEELSEEGYLAKVKSLLNKDLSLENGTHTLTQKANGCPANGSRPTWRAEMADSNRSPRSRPKPRGPRRSKSDSDTLFETSPSSVATRRTTRQTTITAHFTKGPTKRKPKEESEEGNSAESAAEERDQDKKRRVVDTESGAAAAVEKLEEVTAGTQLGPEEPCEQEDDNRSLRRHTRELSLRRKSKEDPDREARPETHLDEDEDGKKDKRSSRPRSQPRDPAAKRRPKEAEPEQVAPETPEDRDEDEREEKRRKTTRKKLESHTVPVQSRSERKAAQSKSVIPKINSPKCPECGQHLDDPNLKYQQHPEDAVDEPQMLTSEKLSIYDSTSTWFDTYEDSPMHRFTSFSVYCSRGHLCPVDTGLIEKNVELYFSGCAKAIHDENPSMEGGINGKNLGPINQWWLSGFDGGEKVLIGFSTAFAEYILMEPSKEYEPIFGLMQEKIYISKIVVEFLQNNPDAVYEDLINKIETTVPPSTINVNRFTEDSLLRHAQFVVSQVESYDEAKDDDETPIFLSPCMRALIHLAGVSLGQRRATRRVMGATKEKDKAPTKATTTKLVYQIFDTFFSEQIEKYDKEDKENAMKRRRCGVCEVCQQPECGKCKACKDMVKFGGTGRSKQACLKRRCPNLAVKEADDDEEADDDVSEMPSPKKLHQGKKKKQNKDRISWLGQPMKIEENRTYYQKVSIDEEMLEVGDCVSVIPDDSSKPLYLARVTALWEDKNGQMMFHAHWFCAGTDTVLGATSDPLELFLVGECENMQLSYIHSKVKVIYKAPSENWAMEGGTDPETTLPGAEDGKTYFFQLWYNQEYARFESPPKTQPTEDNKHKFCLSCIRLAELRQKEMPKVLEQIEEVDGRVYCSSITKNGVVYRLGDSVYLPPEAFTFNIKVASPVKRPKKDPVNETLYPEHYRKYSDYIKGSNLDAPEPYRIGRIKEIHCGKKKGKVNEADIKLRLYKFYRPENTHRSYNGSYHTDINMLYWSDEEAVVNFSDVQGRCTVEYGEDLLESIQDYSQGGPDRFYFLEAYNSKTKNFEDPPNHARSPGNKGKGKGKGKGKGKHQVSEPKEPEAAIKLPKLRTLDVFSGCGGLSEGFHQAGISETLWAIEMWDPAAQAFRLNNPGTTVFTEDCNVLLKLVMAGEVTNSLGQRLPQKGDVEMLCGGPPCQGFSGMNRFNSRTYSKFKNSLVVSFLSYCDYYRPRFFLLENVRNFVSYRRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEIQNGASNSEIPYNGEPLSWFQRQLRGSHYQPILRDHICKDMSPLVAARMRHIPLFPGSDWRDLPNIQVRLGDGVIAHKLQYTFHDVKNGYSSTGALRGVCSCAEGKACDPESRQFSTLIPWCLPHTGNRHNHWAGLYGRLEWDGFFSTTVTNPEPMGKQGRVLHPEQHRVVSVRECARSQGFPDSYRFFGNILDRHRQVGNAVPPPLAKAIGLEIKLCLLSSARESASAAVKAKEEAATKD

SEQ ID NO: 5 - RNA, 22 nt, Homo sapiens
ucagugcacuacagaacuuugu

SEQ ID NO: 6 - RNA, 22 nt, Mus musculus
ucagugcacuacagaacuuugu

SEQ ID NO: 7 - RNA, 68 nt, Homo sapiens
gaggcaaaguucugagacacuccgacucugaguaugauagaagucagugcacuacagaacuuugucuc

SEQ ID NO: 8 - RNA, 99 nt, Mus musculus
agccaguuuggucuuuugagacaaaguucugagacacuccgacucugaguaugauagaagucagugcacuacagaacuuugucucuagaggcugugguc

SEQ ID NO: 9 - DNA, 21 bp, Artificial Sequence
  Synthetic Dnmt-1 siRNA sense strand. Combined DNA/RNA.
gguagagaguuacgacgaatt

SEQ ID NO: 10 - DNA, 21 bp, Artificial Sequence
  Synthetic Dnmt-1 siRNA antisense strand. Combined DNA/RNA.
uucgucguaacucucuacctg

SEQ ID NO: 11 - DNA, 21 bp, Artificial Sequence
  Synthetic Dnmt-1 siRNA sense strand. Combined DNA/RNA.
caacggauccuaucacacutt

SEQ ID NO: 12 - DNA, 21 bp, Artificial Sequence
  Synthetic Dnmt-1 siRNA antisense strand. Combined DNA/RNA.
agugugauaggauccguugta

(19) United States
(12) Patent Application Publication
(10) Pub. No.: US 2015/0218514 A1
(43) Pub. Date: Aug. 6, 2015

(54) PROMOTER OF DIFFERENTIATION FROM HEPATIC PROGENITOR CELL INTO HEPATIC CELL, AND USE THEREOF

(71) Applicants: NATIONAL CANCER CENTER, Tokyo (JP); DS PHARMA BIOMEDICAL CO., LTD., Suita-shi, Osaka (JP)

(72) Inventors: Takahiro Ochiya, Tokyo (JP); Luc Gailhouste, Tokyo (JP)

(73) Assignees: NATIONAL CANCER CENTER, Tokyo (JP); DS PHARMA BIOMEDICAL CO., LTD., Suita-shi, Osaka (JP)

(21) Appl. No.: 14/420,549

(22) PCT Filed: Aug. 9, 2013
(86) PCT No.: PCT/JP2013/071726

§ 371 Date: Feb. 9, 2015

(30) Foreign Application Priority Data
Aug. 9, 2012 (JP) 2012-177171
Jan. 11, 2013 (JP) 2013-003365

(51) Int. Cl. C12N 5/071; C12Q 1/68; G01N 33/50; C12N 15/113

(52) U.S. Cl. CPC C12N 5/067; C12N 15/1137; C12Q 1/6883; G01N 33/5067; C12N 2310/14; C12N 2310/141; C12Q 2600/158; C12Q 2600/154; C12Q 2600/142

(57) ABSTRACT

The present invention provides a promoter of differentiation from a hepatic progenitor cell into a hepatocyte, which contains a substance that suppresses expression of Dnmt-1 or a substance that inhibits the function of Dnmt-1 as an active ingredient, and a method of producing a hepatocyte (preferably hepatocyte with high maturity) from a hepatic progenitor cell.

### TECHNICAL FIELD

The present invention relates to a promoter of differentiation from hepatic progenitor cell or hepatoblastoma into hepatocyte, a method of producing hepatocyte from hepatic progenitor cell or hepatoblastoma and the like.

### BACKGROUND ART

In new drug development processes, tests utilizing hepatocytes are performed to predict hepatotoxicity or liver metabolite. As hepatocytes, human primary cultured hepatocytes are generally used. However, since human primary cultured hepatocytes are associated with problems that (1) a good lot is difficult to obtain in Japan since they depend on the import from abroad, (2) since primary cells do not grow, the number of samples in the same lot is limited, and the like, which prevent stable supply and continuous use thereof. Therefore, a method of obtaining a hepatocyte by differentiation induction of a stem cell (artificial pluripotent stem cell (iPS cell), embryonic stem cell (ES cell), mesenchymal stem cell) showing superior growth has been demanded.

As a method for inducing differentiation of stem cell into hepatocyte, a method including stepwise introduction of plural genes into iPS cell (non-patent document 1), a method including culturing mesenchymal stem cells in a special differentiation induction medium (non-patent document 2) and the like are known. However, it is known that a hepatocyte prepared by differentiation induction of stem cell by a conventional method shows low drug-metabolizing enzyme activity (CYP activity) and the like as compared to human primary cultured hepatocytes and maturation is insufficient, and a hepatocyte having high maturity has not been obtained as yet.

At present, the RNA interference (RNAi) technique is frequently utilized for the research of life sciences, and its usefulness has widely been confirmed. RNAi refers to a phenomenon wherein mRNA is degraded by double stranded RNA in a sequence-specific manner, thereby suppressing the gene expression. Ever since a report in 2001 teaching that low molecule double stranded RNA with 21 bases can mediate RNAi in the cell of a mammal (non-patent document 3), siRNA (small interference RNA) has been frequently used as a method of suppressing expression of a target gene. siRNA is expected to be applicable to pharmaceutical products, and treatment of various refractory diseases including cancer.

microRNA (miRNA) is a short chain RNA molecule composed of about 22 base pairs, which was found in nematode in 1993, which is known to bind to a complementary sequence in the 3′ untranslated region of messenger RNA of the target gene to suppress the translation thereof and regulate various biological phenomena. It has been reported that human contains 1921 kinds and mouse contains 1157 kinds of microRNAs (http://www.mirbase.org/). Interestingly, plural target genes exist for one microRNA, and they are often involved in the same physiological function and the same signal transduction pathway.

Therefore, variation in the expression of only one kind of microRNA can cause a dynamic change in the physiological action.

It has been suggested heretofore that microRNA plays an important role in the differentiation of hepatocytes. Non-patent document 4 discloses that stable overexpression of miR-122 in HepG2 (cell line derived from human liver cancer but used due to similarity to immature/undifferentiated hepatoblast) suppresses cell proliferation, and a further increase in the copy number of miR-122 changes the cell morphology as well as suppresses cell proliferation, and increases expression of cytochrome P450 family genes that are expressed in mature hepatocytes, thus suggesting that miR-122 plays an important role in the differentiation of hepatocytes in the development of the liver. Non-patent document 5 discloses that transfection of bipotent murine embryonic liver (BMEL) cells with miR-122 mimics increases expression of hepatocyte-specific gene, and differentiation of hepatocytes is promoted in transgenic mouse with increased miR-122 expression, and shows that miR-122 positively regulates differentiation into hepatocytes. Non-patent document 6 discloses that transfection of resident liver stem cells (RLSC-derived hepatocytes) differentiated into hepatocytes with an inhibitor of miRs-200a, b or c results in a mesenchymal-like morphology, increased expression of mesenchymal marker and an increase in the cell expressing stem cell markers, thus suggesting involvement of these microRNAs in the differentiation into hepatocytes. Non-patent document 7 discloses the analysis results of miRNA expression patterns of hepatocytes differentiated from human embryonic stem cells (hESCs) via definitive endoderm (DE) by a known method, which suggest that various miRNAs function in a cell line specific manner in the process of differentiation from hESCs into hepatocytes.

While involvement of miR-148a in the growth of some types of cancer is suggested, whether it is involved in the differentiation into hepatocytes is not known at all.

It is heretofore known that the mRNA level of CYP gene increases by treating HepG2 cells for 2 days with 5-aza-2-deoxycytidine that inhibits DNA methylation caused by Dnmt-1 (non-patent document 8). However, an increase in the CYP expression at the protein level has not been reported. On the other hand, it is known by study using HepG2 cells that miR-148a suppresses translation of PXR that promotes gene transcription of CYP3A4 (non-patent document 9). Furthermore, it has been reported that a substance inhibiting DNA methylation caused by Dnmt-1 increases expression of miR-148a (non-patent document 10). Since the expression of miR-148a increases by the inhibition of DNA methylation caused by Dnmt-1, expression of PXR is suppressed, and an increase in the mRNA expression amount of CYP gene may not be reflected on the increase at the protein level (non-patent document 11).

In addition, 5-aza-2-deoxycytidine that inhibits DNA methylation caused by Dnmt-1 has been reported to show high cytotoxicity and induce apoptosis (non-patent document 12).

### DOCUMENT LIST

### Non-Patent Documents

non-patent document 1: Molecular Therapy, Vol. 20(1), p. 127-137 (2012).
non-patent document 2: Biochemical and Biophysical Research Communications, Vol. 328(1), p. 258-264(2005).
non-patent document 3: Nature, Vol. 411, p. 494-8 (2001).
non-patent document 4: HEPATOLOGY, Vol. 52(4), p. 1431-1442 (2010).
non-patent document 5: GASTROENTEROLOGY, Vol. 142(1), p. 119-129 (2012).
non-patent document 6: Cell Death Differ, Vol. 19(6), p. 937-946 (2012) (doi: 10.1038/cdd.2011.175. Epub 2011 Dec. 2, 1-10).
non-patent document 7: Hepatology Research, Vol. 41, p. 170-183 (2011).
non-patent document 8: BMC Genomics. 2006, 7; 181.
non-patent document 9: Journal of Biological Chemistry, Vol. 283, p. 9674-9680 (2008).
non-patent document 10: Proceedings of the National Academy of Sciences of the United States of America, Vol. 105, p. 13556-13561 (2008).
non-patent document 11: Epigenetic regulation of pharmacokinetics-associated genes expression by DNA methylation Atsushi Miyajima, Graduate School of Pharmaceutical Sciences, Chiba University, i-yaku-haku “KOU” No. 93.
non-patent document 12: Cancer Res, Vol. 66(5), p. 2794-2800(2006).

### SUMMARY OF THE INVENTION

### Problems to be Solved by the Invention

It is known that hepatocytes produced from stem cells by differentiation induction by a conventional method have problems in that the drug-metabolizing enzyme activity (CYP activity) and the like are low and maturation is insufficient, as compared to human primary cultured hepatocytes. An object of the present invention is to provide a method of producing hepatocytes (preferably hepatocyte with high maturity) in a short period by promoting differentiation from hepatic progenitor cells into hepatocytes.

### Means of Solving the Problems

The present inventors have conducted intensive studies in an attempt to achieve the above-mentioned object and found that miR-148a is highly expressed in adult mouse hepatic tissues as compared to mouse embryonic hepatic tissue, newborn mouse hepatic tissue and mouse liver cancer-derived cell line (Hepa1-6), and that miR-148a is highly expressed in human normal hepatic tissue as compared to human liver cancer-derived cell lines (Huh-7, HepG2 and Hep3B). miR-148a precursor was introduced into hepatic progenitor cells and they were induced to differentiate into hepatocytes by culturing in a differentiation induction medium. As a result, the expression levels of albumin, glucose-6-phosphatase (G6pc), tyrosine aminotransferase (Tat), cytochrome P450 (Cyp) and miR-122, which are indices of liter function expression, remarkably increased in a short period as compared to the absence of introduction of miR-148a precursor. Furthermore, they have found that the expression level of Dnmt-1, which is a DNA methyltransferase, decreases after differentiation induction from hepatic progenitor cells into hepatocytes, namely, in inverse correlation with miR-148a expression level, and the expression level of Dnmt-1 further decreases in hepatic progenitor cells introduced with miR-148a precursor. These findings show that the expression of Dnmt-1 can be suppressed by utilizing miR-148a, whereby hepatocyte with high maturity and showing increased expression of hepatocyte marker can be obtained in a short period. In fact, when hepatic progenitor cell introduced with siRNA (siDnmt-1) that suppresses expression of Dnmt-1 was induced to differentiate into hepatocyte, the expression levels of albumin, G6pc, Tat and Cyp remarkably increased as compared to when control siRNA that does not suppress expression of Dnmt-1 was introduced. From the above findings, the present inventors have found that differentiation into hepatocyte can be promoted by suppressing expression of Dnmt-1 and conducted further studies, which resulted in the completion of the present invention.

Accordingly, the present invention relates to the following.

[1] A promoter of differentiation from hepatic progenitor cell into hepatocyte, which comprises a substance that suppresses expression of Dnmt-1 or a substance that inhibits function of Dnmt-1 as an active ingredient.
[2] The differentiation promoter of [1], wherein the substance that suppresses expression of Dnmt-1 is a nucleic acid that suppresses expression of Dnmt-1 or a precursor thereof.
[3] The differentiation promoter of [2], wherein the nucleic acid that suppresses expression of Dnmt-1 or precursor thereof is miRNA for mRNA of Dnmt-1 gene or a precursor thereof, or siRNA for mRNA of Dnmt-1 gene or a precursor thereof.
[4] The differentiation promoter of [3], wherein the nucleic acid that suppresses expression of Dnmt-1 or precursor thereof is miR-148a or a precursor thereof.
[5] The differentiation promoter of [1], wherein the substance that inhibits function of Dnmt-1 is a substance that inhibits DNA methylation caused by Dnmt-1.
[6] The differentiation promoter of [1], wherein the substance that inhibits DNA methylation caused by Dnmt-1 is a cytidine analog or a salt, solvate, hydrate, precursor or derivative thereof.
[7] The differentiation promoter of [5], wherein the substance that inhibits DNA methylation caused by Dnmt-1 masks a Dnmt-1 target sequence.
[8] The differentiation promoter of [5], wherein the substance that inhibits DNA methylation caused by Dnmt-1 blocks an active site of Dnmt-1.
[9] The differentiation promoter of any of [1]-[8], wherein the hepatic progenitor cell is a primary cultured cell or a cell induced to differentiate from a stem cell.
[10] The differentiation promoter of [9], wherein the hepatic progenitor cell is a primary cultured cell.
[11] The differentiation promoter of [9], wherein the hepatic progenitor cell is a cell induced to differentiate from an embryonic stem cell, an artificial pluripotent stem cell or a mesenchymal stem cell.
[12] A method of producing a hepatocyte from a hepatic progenitor cell, comprising the following steps:
(1) a step of suppressing expression of Dnmt-1 or inhibiting function of Dnmt-1 in a hepatic progenitor cell by contacting the hepatic progenitor cell with the substance that suppresses expression of Dnmt-1 or substance that inhibits function of Dnmt-1 of any of [1]-[9]; and
(2) a step of inducing differentiation into a hepatocyte by culturing the hepatic progenitor cell obtained in the aforementioned step (1), wherein expression of Dnmt-1 is suppressed or function of Dnmt-1 is inhibited.
[13] The method of [12], wherein the hepatic progenitor cell is a primary cultured cell a cell induced to differentiate from or a stem cell.
[14] The method of [13], wherein the hepatic progenitor cell is a primary cultured cell.
[15] The method of [13], wherein the hepatic progenitor cell is a cell induced to differentiate from an embryonic stem cell, an artificial pluripotent stem cell or a mesenchymal stem cell.
[16] A hepatocyte produced by the method of any of [12]-[15]. [17] A method of evaluating metabolism of a test compound, comprising the steps of the following (a) and (b):
(a) a step of contacting the hepatocyte of [16] with a test compound; and
(b) a step of measuring the metabolism of the test compound contacted with the hepatocyte in the aforementioned step (a).
[18] A method of evaluating hepatotoxicity of a test compound, comprising the steps of the following (a) and (b):
(a) a step of contacting the hepatocyte of [16] with a test compound; and
(b) a step of measuring the level of disorder of the hepatocyte contacted with the test compound in the aforementioned step (a).
[19] A promoter of differentiation from hepatoblastoma into hepatocyte, which comprises a substance that suppresses expression of Dnmt-1 or a substance that inhibits function of Dnmt-1 as an active ingredient.
[20] A method of producing a hepatocyte from a hepatoblastoma, comprising the following steps:
(1) a step of suppressing expression of Dnmt-1 or inhibiting function of Dnmt-1 in a hepatoblastoma by contacting the hepatoblastoma with the substance that suppresses expression of Dnmt-1 or substance that inhibits function of Dnmt-1 of any of [1]-[9]; and
(2) a step of inducing differentiation into a hepatocyte by culturing the hepatoblastoma obtained in the aforementioned step (1), wherein expression of Dnmt-1 is suppressed or function of Dnmt-1 is inhibited for not less than 7 days.

### Effect of the Invention

According to the present invention, differentiation from a hepatic progenitor cell or hepatoblastoma into a hepatocyte can be promoted. As a result, a hepatocyte having higher maturity than hepatocytes produced by inducing differentiation from a stem cell by a conventionally-known method can be provided in a short period.

### BRIEF DESCRIPTION OF THE DRAWINGS

FIG. 1 shows changes in the morphology of hepatic progenitor cells after differentiation induction. The cultured cells reached confluent 3 days after culture and the cell morphology was quadrate (a). Five days after culture, the cell morphology became oval (b). Twelve days after culture, the cell morphology became that of matured hepatocyte (c and d). Scale bar: 200 μm (a, c), 50 μm (b, d).

FIG. 2 shows the expression levels of miR-148a in hepatic tissues or liver cancer-derived cell lines of human (right panel) and mouse (left panel). mean±SEM (n=10).

FIG. 3 shows changes in the expression level of miR-148a after differentiation induction in hepatic progenitor cells. mean±SEM (n=3).

FIG. 4 shows changes in the expression level of miR-148a after differentiation induction in hepatic progenitor cells introduced with miR-148a precursor. The black bar shows the expression level of hepatic progenitor cells not introduced with miR-148a precursor, and the gray bar shows the expression level of hepatic progenitor cells introduced with miR-148a precursor. mean±SEM (n=3).

FIG. 5 shows changes in the expression level of albumin, G6pc, Tat, Cyp17a1 and miR-122 after differentiation induction in hepatic progenitor cells introduced with miR-148a precursor. The bar on the left side shows the expression level in hepatic progenitor cells not introduced with miR-148a, and the bar on the right side shows the expression level in hepatic progenitor cells introduced with miR-148a precursor. mean±SEM (n=3).

FIG. 6 shows changes in the expression level of Dnmt-1 after differentiation induction in hepatic progenitor cells. mean±SEM (n=3).

FIG. 7 shows the expression level of Dnmt-1 on day 4 (D4) and day 5 (D5) after the start of culture in hepatic progenitor cells introduced with miR-148a precursor. The bar on the left side shows the expression level in hepatic progenitor cells not introduced with miR-148a, and the bar on the right side shows the expression level in hepatic progenitor cells introduced with miR-148a precursor. mean±SEM (n=3).

FIG. 8 shows changes in the expression level of Dnmt-1, albumin, G6pc, Tat and Cyp17a1 on day 6 after the start of culture in hepatic progenitor cell introduced with siRNA (siDnmt-1) that suppresses expression of Dnmt-1. “siCtrl” shows the expression level in hepatic progenitor cell introduced with siRNA (siCtrl) that does not suppress expression of Dnmt-1, and “siDnmt-1” shows the expression level in hepatic progenitor cell introduced with siDnmt-1 (a and b show two kinds of siRNAs (siDnmt-1) with Dnmt-1 as target gene, whose base sequences are different). mean±SEM (n=3).

FIG. 9 shows changes in the expression level of miR-148a after differentiation induction in hepatic progenitor cells cultured in a medium added with substance that inhibits DNA methylation caused by Dnmt-l. mean±SEM (n=3).

FIG. 10 shows changes in the expression level of hepatocyte marker after differentiation induction in hepatic progenitor cells cultured in a medium added with substance that inhibits DNA methylation caused by Dnmt-1. mean±SEM (n=3).

FIG. 11 shows the expression levels of (A) albumin (ALB) and (B) Cyp3a4 in human hepatic progenitor cell cultured in a medium alone (control), human hepatic progenitor cell introduced with miR-148a precursor (miR-148a), and human hepatic progenitor cell introduced with miRNA without function (miCtrl) after differentiation induction. The bar on the left side shows the expression level in cells introduced with control, the middle bar shows the expression level in cells introduced with miCtrl, and the bar on the right side shows the expression level in cells introduced with miR-148a.

FIG. 12 shows observation, under an optical microscope, of human hepatic progenitor cells introduced with miR-148a precursor. A cell having a morphology with multiple nuclei and a clear cell boundary, which are specific to mature hepatocytes, is shown enclosed with a circle in the figure. scale bar: 50 μm.

FIG. 13 shows the cell morphology of HepG2 cells after treating with 5-aza-2′-deoxycytidine (2.5 μM) which is a substance that inhibits DNA methylation caused by Dnmt-1. Cells cultured under the same conditions except the treatment with 5-aza-2′-deoxycytidine were used as a control.

FIG. 14 shows the results of PAS staining of HepG2 cells after treatment with 5-aza-2′-deoxycytidine (2.5 μM). Cells cultured under the same conditions except the treatment with 5-aza-2′-deoxycytidine were used as a control.

FIG. 15 shows changes in the expression level of hepatocyte marker in HepG2 cells after treating with 5-aza-2′-deoxycytidine(5-aza) (2.5 μM). Cells cultured under the same conditions except the treatment with 5-aza-2′-deoxycytidine were used as a control.

FIG. 16 shows the measured activity of CYP enzyme in HepG2 cells after treatment with a substance that inhibits DNA methylation caused by Dnmt-1 (5-aza-2′-deoxycytidine(5-aza) or RG108), or an inhibitory nucleic acid for Dnmt-1 (miR-148a or siDnmt1). Cells cultured under the same conditions except the treatment with the inhibitory substance and the inhibitory nucleic acid were used as a control.

FIG. 17 shows the measured activity of CYP enzyme (CYP1A2 or CYP3A4) in HepG2 cells after treatment with 5-aza-2′-deoxycytidine(5-aza) (2.5 μM) followed by enzyme induction with dexamethasone (25 μM). Cells cultured under the same conditions except the treatment with 5-aza-2′-deoxycytidine were used as a control.

### DESCRIPTION OF EMBODIMENTS

1. Differentiation Promoter from Hepatic Progenitor Cell or Hepatoblastoma into Hepatocyte

The present invention provides a promoter of differentiation from a hepatic progenitor cell into a hepatocyte, which contains, as an active ingredient, a substance that suppresses expression of Dnmt-1 or a substance that inhibits function of Dnmt-1. In a further aspect, the present invention provides a promoter of differentiation from a hepatoblastoma into a hepatocyte, which contains, as an active ingredient, a substance that suppresses expression of Dnmt-1 or a substance that inhibits function of Dnmt-1. The promoter of differentiation from a hepatic progenitor cell into a hepatocyte, and the promoter of differentiation from a hepatoblastoma into a hepatocyte above are collectively referred to as the promoter of the present invention. Dnmt-1 is one of the DNA methyltransferases of mammals. The present invention is based on the finding that the expression of hepatocyte marker is promoted in differentiation induction from a hepatic progenitor cell or hepatoblastoma into a hepatocyte by suppressing the expression of Dnmt-1 or inhibiting the function of Dnmt-1.

The promoter of the present invention after incorporation into a cell shows an activity to suppress expression of Dnmt-1, or inhibit the function of Dnmt-1 activity. The activity to suppress expression of Dnmt-1 can be examined by using a transformant transfected with Dnmt-1 gene, Dnmt-1 gene expression system in vivo or in vitro, or Dnmt-1 translation system Dnmt-1 in vivo or in vitro. In addition, the activity to inhibit function of Dnmt-1 can be examined by using in vivo or in vitro DNA methyltransferase activity measurement system of Dnmt-1 known per se. For example, the promoter of the present invention can be judged to have a Dnmt-1 function inhibitory activity when a cell that expresses Dnmt-1 is cultured in the presence of the promoter of the present invention, the methylation states of the parent cell DNA and the daughter cell DNA are examined, and the methylation state of the parent cell DNA is not transmitted to the daughter cell DNA.

The promoter of the present invention is introduced into a hepatic progenitor cell or hepatoblastoma and promotes differentiation into a hepatocyte. In the present invention, the “hepatocyte” is a cell that expresses at least one kind of hepatocyte markers (e.g., albumin, glucose-6-phosphatase (G6pc), tyrosine aminotransferase (Tat), cytochrome P450 (Cyp), miR-122 etc.) (preferably, 4 kinds of albumin, G6pc, Tat and Cyp, more preferably 5 kinds of albumin, G6pc, Tat, Cyp and miR-122). To “promote differentiation into hepatocyte” means to increase the expression level of at least one kind of hepatocyte markers (e.g., albumin, glucose-6-phosphatase (G6pc), tyrosine aminotransferase (Tat), cytochrome P450 (Cyp), miR-122 etc.) (preferably, 4 kinds of albumin, G6pc, Tat and Cyp, more preferably, 5 kinds of albumin, G6pc, Tat, Cyp and miR-122) after a lapse of a given number of days (e.g., 1 day, 2 days, 3 days, 4 days, 5 days, 6 days, 7 days etc.) from the start of differentiation induction, as compared to differentiation induction of hepatic progenitor cell or hepatoblastoma not introduced with the promoter of the present invention. In this case, differentiation induction is performed by culturing the cells in William's E medium (Invitrogen Corporation) added with 2 mM L-glutamine solution (Invitrogen Corporation), 50 U/mL penicillin-50 μg/mL streptomycin solution (Invitrogen Corporation), 10% fetal bovine serum (Thermo Fisher Scientific K.K.), 5 μg/mL insulin (Sigma-Aldrich Corporation), 25 ng/mL epidermal growth factor (EGF) (Sigma-Aldrich Corporation), 25 ng/mL hepatocyte growth factor (HGF) (Peprotech), 12.5 ng/mL oncostatin M (Sigma-Aldrich Corporation), 5×10⁻⁷M hydrocortisone 21-hemisuccinate sodium salt (Sigma-Aldrich Corporation), 10⁻⁷ M dexamethasone (Sigma-Aldrich Corporation) for 48 hr, and thereafter culturing the cells in a medium free of epidermal growth factor (EGF) and fetal bovine serum. Alternatively, in a further aspect, differentiation induction of hepatoblastoma can be performed by, for example, continuous cultivation in a DMEM medium (Invitrogen Corporation) added with 2 mM L-glutamine solution (Invitrogen Corporation), 50 U/mL penicillin-50 μg/mL streptomycin solution (Invitrogen Corporation), 10% fetal bovine serum (Thermo Fisher Scientific K.K.). The expression level of a hepatocyte marker can be measured by a method known per se. For example, total RNA is extracted from a cell, and the mRNA level or miRNA level of the hepatocyte marker may be quantified by a known method such as real-time PCR method and the like.

In the present invention, the hepatic progenitor cell means a cell having proliferative capacity and an ability to differentiate into two directions of hepatocyte and bile duct epithelial cell, for which E-cadherin has been identified as a surface antigen marker. The hepatic progenitor cell also includes hepatic stem cells and hepatoblasts.

The hepatic progenitor cell used in the present invention is not particularly limited as long as it has the above-mentioned properties, and is preferably a non-tumor cell.

The hepatic progenitor cell used in the present invention is generally a cell of a mammal (e.g., rat, mouse, guinea pig, rabbit, sheep, horse, swine, bovine, monkey, human, preferably human).

The promoter of the present invention can promote differentiation into hepatocyte as long as the cell is a hepatic progenitor cell, irrespective of the method of obtaining same. Hepatic progenitor cell can be obtain by a method known per se, and examples thereof include, but are not limited to, cells induced to differentiate from primary cultured cell obtained from a living organism, stem cell (embryonic stem cell (ES cell), induced pluripotent stem cell (iPS cell), mesenchymal stem cell etc.) and the like.

In one embodiment, hepatic progenitor cell is a primary cultured cell. For example, hepatic progenitor cell can be isolated by sorting out E-cadherin positive cells from the hepatocyte group contained in the liver (preferably, viviparous stage liver) isolated from a living organism. More specifically, hepatic progenitor cell can be isolated by the following steps which are not limitative: collecting hepatic tissues from embryo, cutting them into small pieces with surgical scissors etc., and treating them with an enzyme solution containing protease (e.g., liberase (manufactured by Roche Applied Science) etc.). The dispersed cells are washed with a medium (e.g., basal medium for hepatocyte) to give hepatic parenchymal cell. The obtained hepatic parenchymal cells are contacted with a biotin-labeled anti-E-cadherin antibody (e.g., manufactured by eBioscience), and the cells labeled with the biotin-labeled antibody are recovered using EasySep Mouse Biotin Positive Selection Kit (manufactured by STEMCELL Technologies) and the like. The recovered cells are hepatic progenitor cells.

In another embodiment, the hepatic progenitor cell is a cell induced to differentiate from a stem cell. Stem cell includes embryonic stem cells (ES cell), induced pluripotent stem cell (iPS cell) and mesenchymal stem cell.

Examples of the ES cell include an embryonic stem cell of a mammal and the like, which is established by cultivating early embryo before implantation, an embryonic stem cell established by cultivating an early embryo prepared by nuclear transplantation of the nucleus of a somatic cell, and an embryonic stem cell obtained by alteration of its gene(s) on chromosome(s) by a known genetic engineering method.

To be specific, examples of the embryonic stem cell include embryonic stem cells established from an inner cell mass, single blastomere and the like constituting an early embryo, EG cell established from a primordial germ cell, a cell isolated from a cell population (e.g., primitive ectoderm) having multipotency of an early embryo before implantation, a cell obtained by cultivating said cell and the like. Also, these embryonic stem cells may be those distinguishable to have reached the corresponding differentiation stage by using expression of a marker gene as an index, by inflame knock-in of the marker gene (e.g., fluorescence protein such as GFP and the like) into a gene encoding the differentiation marker by a known method.

Embryonic stem cells are available from predetermined institutions, and commercially available products can also be purchased. For example, KhES-1, KhES-2 and KhES-3, which are human embryonic stem cells, are available from Institute for Frontier Medical Sciences, Kyoto University.

Fused ES cells obtained by cell fusion of ES cell and somatic cell are also included in the embryonic stem cells to be used for the method of the present invention.

Induced pluripotent stem cell (iPS cell) is obtained by reprogramming somatic cell (e.g., fibroblast, skin cell etc.) by introduction of a reprogramming factor. Induced pluripotent stem cell was found for the first time by a method including introducing reprogramming factors consisting of Oct3/4, Sox2, Klf4 and c-Myc into a somatic cell (e.g., fibroblast, skin cell etc.) (Cell, 126: p. 663-676, 2006). Thereafter, many researchers are working on various improvements in the combinations of reprogramming factors and introduction method of the factors, and various production methods of induced pluripotent stem cells have been reported. The induced pluripotent stem cell in the present invention also includes cells produced by such methods.

Mesenchymal stem cell is a somatic stem cell derived from mesenchyme, and has differentiation potency into a cell belonging to mesenchyme. It is also shown to have plasticity enabling differentiation into non-mesodermal tissues such as ectoderm-derived cells, endoderm-derived cells and the like. Mesenchymal stem cell includes, for example, bone marrow mesenchymal cell and cord blood-derived stem cell. While mesenchymal stem cell is considered to exist in any mesenchymal tissues, bone marrow mesenchymal stem cell can be easily collected by bone marrow puncture from among the mesenchymal tissues, and culture techniques have been established. Bone marrow mesenchymal stem cell is included in bone marrow interstitial cells, and bone marrow interstitial cell is one kind of cell that supports hematopoietic cell to be the main cell in the bone marrow. On the other hand, cord blood is the blood contained in the umbilical cord as an embryo side tissue connecting embryo and mother. While it is known that a large amount of cord blood-derived stem cells (hematopoietic stem cells) is contained in the cord blood, it has also been clarified that mesenchymal stem cells (cord blood-derived mesenchymal stem cells), which are somatic stem cells other than hematopoietic stem cell, are also present.

Induction of differentiation from stem cell into hepatic progenitor cell can be performed, for example, by culturing the cell in a culture medium added with activin A, and culturing the cell in a culture medium added with bone morphogenic protein (BMP) and fibroblast growth factor (FGF), and the like.

Hepatoblastoma is a malignant tumor developed from immature hepatoblast. Hepatoblastoma may be established, and examples of the established cell line include HepG2.

Dnmt-1 is a molecule known already, and is known to have a DNA methyltransferase activity. Conventionally, the correlation between the Dnmt-1 expression level in liver cancer cells and the malignancy level of liver cancer has been suggested (International Journal of Cancer, Volume 105, Issue 4, pages 527-532, 1 Jul. 2003); however, the correlation between Dnmt-1 and hepatocyte differentiation is not known at all.

Amino acid sequence of Dnmt-1 and base sequence encoding same are published in several kinds of animals. For example, a base sequence and an amino acid sequence of human Dnmt-1 have been published as NCBI accession No. NM_{—}001130823.1 (renewed on Jun. 17, 2012) (SEQ ID NO: 1) and NP_{—}001124295.1 (renewed on Jun. 17, 2012) (SEQ ID NO: 2), and the base sequence and amino acid sequence of mouse Dnmt-1 have been published as NCBI accession No. NM_{—}010066.4 (renewed on May 5, 2012) (SEQ ID NO: 3) and NP_{—}034196.5 (renewed on May 5, 2012) (SEQ ID NO: 4), respectively. Sequence information of Dnmt-1 ortholog of other mammal and naturally-occurring variants can be obtained from public databases (e.g., HomoloGene (http://www.ncbi.nlm.nih.gov/HomoloGene/) and UniProtKB/Swiss-Protdatabase etc.) well known in the pertinent technical field.

The function of Dnmt-1 is known to be introduction of methylation state of the original DNA (Parent Strand) into a new DNA (Daughter strand) by DNA methyltransferase activity in the DNA replication process.

In the present invention, “a substance that suppresses expression of Dnmt-1” may act in any stage such as transcription level of Dnmt-1 gene encoding Dnmt-1, post-transcription regulation level, translation level into Dnmt-1, post-translational modification level and the like. Therefore, Examples of the substance that suppresses expression of Dnmt-1 include a substance that inhibits transcription of Dnmt-1 gene (e.g., antigen), a substance that inhibits processing of initial transcription product to mRNA, a substance that inhibits transportation of mRNA to the cytoplasm, a substance that inhibits translation of mRNA into Dnmt-1 (e.g., antisense nucleic acid, miRNA) or degrades mRNA (e.g., siRNA, ribozyme, miRNA), a substance that inhibits post-translational modification of initial translation product and the like. While any substance that acts on any stage can be preferably used, a substance that complementarily binds to mRNA to inhibit translation into Dnmt-1 or degrade mRNA is preferable.

Preferable examples of the substance that specifically inhibits translation of mRNA of Dnmt-1 gene into Dnmt-1 (or degrades mRNA) include a nucleic acid containing a base sequence complementary or substantially complementary to the base sequence of the mRNA, or a part thereof.

In the present invention, nucleic acid is RNA, chimeric nucleic acid of RNA and DNA (hereinafter to be referred to as chimeric nucleic acid) or hybrid nucleic acid. As used herein, chimeric nucleic acid means that a single stranded or double stranded nucleic acid contains RNA and DNA in a single nucleic acid, and hybrid nucleic acid refers to a double stranded nucleic acid having RNA or chimeric nucleic acid for one chain and DNA or chimeric nucleic acid for the other chain.

In the present invention, nucleic acid is single stranded or double stranded. Embodiments of double strand include double stranded RNA, double stranded chimeric nucleic acid, RNA/DNA hybrid, RNA/chimeric nucleic acid hybrid, chimeric nucleic acid/chimeric nucleic acid hybrid, and chimeric nucleic acid/DNA hybrid. In the present invention, nucleic acid is preferably single stranded RNA, single stranded chimeric nucleic acid, double stranded RNA, double stranded chimeric nucleic acid, RNA/DNA hybrid, RNA/chimeric nucleic acid hybrid, chimeric nucleic acid/chimeric nucleic acid hybrid or chimeric nucleic acid/DNA hybrid, more preferably single stranded RNA, single stranded chimeric nucleic acid, double stranded RNA, double stranded chimeric nucleic acid, RNA/DNA hybrid, chimeric nucleic acid/chimeric nucleic acid hybrid or RNA/chimeric nucleic acid hybrid.

The “base sequence substantially complementary to the base sequence of mRNA of Dnmt-1 gene” means a base sequence having complementarity of the level enabling, under physiological conditions of mammals, binding to the target sequence of the mRNA to inhibit translation thereof (or cleave the target sequence), and is specifically, for example, a base sequence having a homology of about not less than 80%, preferably not less than about 90%, more preferably not less than about 95%, particularly preferably not less than about 97%, with a region overlapping with a base sequence completely complementary to the base sequence of the mRNA (i.e., base sequence of complementary chain of mRNA).

The “homology of base sequence” in the present invention can be calculated using homology calculation algorithm NCBI BLAST (National Center for Biotechnology Information Basic Local Alignment Search Tool) under the following conditions (expectancy=10; gap allowed; filtering=ON; match score=1; mismatch score=−3).

The “part of a base sequence complementary or substantially complementary to the base sequence of mRNA of Dnmt-1 gene” is not particularly limited as to its length and position as long as it can specifically bind to mRNA of Dnmt-1 gene, and can inhibit translation of protein from the mRNA (or degrade the mRNA).

Specific examples of the nucleic acid containing a base sequence complementary or substantially complementary to the base sequence of mRNA of Dnmt-1 gene, or a part thereof preferably include the following (a) and (b).

(a) miRNA to mRNA of Dnmt-1 gene or a precursor thereof
(b) siRNA to mRNA of Dnmt-1 gene or a precursor thereof
(a) miRNA to mRNA of Dnmt-1 Gene or a Precursor Thereof

In the present invention, miRNA is a 17-28 base length single stranded RNA, which complementarily binds to target mRNA to inhibit translation of the mRNA (or degrade the mRNA), thus regulating gene expression after transcription. miRNA to mRNA of Dnmt-1 gene may be any miRNA as long as it suppresses expression of Dnmt-1. As a mechanism of suppression of the translation of mRNA of the target gene by miRNA, it is known that mRNA having a base sequence complementary to the 2nd-8th base sequence on the 5′-terminal side of miRNA is recognized as an miRNA target gene by miRNA (Current Biology, 15, R458-R460 (2005)). By this mechanism, expression of the mRNA is suppressed by miRNA. Accordingly, miRNA to mRNA of Dnmt-1 gene preferably has, in the 2nd-8th base sequence on the 5′-terminal side, a base sequence complementary to a base sequence in mRNA of Dnmt-1 gene (preferably in 3′UTR of mRNA). Examples of such miRNA include miR-148a.

miR-148a means an already-known molecule typically called mature miRNA. As used herein, miR-148a includes those derived from any species and examples thereof include molecules derived from human (has-miR-148a (also called has-miR-148a-3p): base sequence shown in SEQ ID NO: 5, registered in miRBase under Accession No. MIMAT0000243), mouse (mmu-miR-148a (also called mmu-miR-148a-3p): base sequence shown inSEQ ID NO: 6, registered in miRBase under Accession No. MIMAT0000516) and the like. In the present invention, miR-148a may be derived from the same or different species from which the cell to be applied with the promoter of the present invention derives, as long as it suppresses expression of Dnmt-1 in the cell to be applied with the promoter of the present invention. In the present invention, a nucleic acid “derived from organism X” means that the base sequence of the nucleic acid contains a base sequence the same or substantially the same as the base sequence of said nucleic acid existing naturally present in the organism X. Being “substantially the same” means that the noted base sequence has an identity of not less than 70% (preferably not less than 80%, more preferably not less than 90%, further preferably not less than 98%, most preferably not less than 99%, with a base sequence of a nucleic acid naturally present in organism X, and maintains the function of the nucleic acid.

miRNA may be introduced into a cell as a mature miRNA, or introduced into a cell as a precursor thereof. A precursor of miRNA means a nucleic acid capable of producing mature miRNA in a cell, as a result of intracellular processing or cleavage of double stranded nucleic acid. Examples of the precursor include pri-miRNA, pre-miRNA and the like. pri-miRNA is a primary transcription product of miRNA gene (single stranded RNA), and generally has a length of about several hundred to several thousand bases. pre-miRNA is a single stranded RNA having a hairpin structure formed by intranuclear processing of pri-miRNA, and generally has a length of 90-110 bases. Since these precursors of miRNAs are naturally-occurring substances, the possibility of being eliminated when introduced in a large amount, or causing cell responses such as interferon response and the like is considered to be low. Examples of the precursor of miRNA to mRNA of Dnmt-1 gene include the precursor of has-miR-148a (base sequence shown in SEQ ID NO: 7, registered in miRBase under Accession No. MI0000253), the precursor of mmu-miR-148a (base sequence shown in SEQ ID NO: 8, registered in miRBase under Accession No. MI0000550) and the like.

In another preferable embodiment, a nucleic acid containing a nucleotide having a similar activity as that of mature miRNA (i.e. activity to suppress expression of Dnmt-1), for example, an miRNA mimic synthesized to mimic endogenous mature miRNA and the like can be used as miRNA. A commercially available one can also be utilized.

miRNA can contain various chemical modifications to improve stability (chemical and/or to enzymatic) and specific activity (affinity for RNA). For example, to prevent decomposition by hydrolase such as nuclease and the like, phosphate residue (phosphate) of each nucleotide constituting miRNA can be substituted by, for example, a chemically modified phosphate residue such as phosphorothioate (PS), methylphosphonate, phosphorodithionate and the like. In addition, the 2′-position hydroxyl group of sugar (ribose) of each nucleotide may be substituted by OR [R═CH₃(2′-O-Me), F(2′-F), CH₂CH₂OCH₃ (2′-O-MOE), CH₂CH₂NHC (NH) NH₂, CH₂CONHCH₃, CH₂CH₂CN etc.]. Furthermore, the base region (pyrimidine, purine) may be chemically modified and, for example, introduction of methyl group or cationic functional group into the 5-position of pyrimidine base, substitution of the 2-position carbonyl group by thiocarbonyl and the like can be mentioned.

The conformation of the sugar region of nucleic acid is dominantly C2′-endo(S type) or C3′-endo(N type); in a single stranded nucleic acid, the two are present in equilibrium and, when a double strand is formed, it is fixed to N type. To impart strong bindability to target RNA, therefore, LNA which is a nucleic acid derivative wherein the conformation of the sugar region is fixed to N type by crosslinking 2′ oxygen and 4′ carbon via a methylene residue (Imanishi, T. et al., Chem. Commun., 1653-9, 2002; Jepsen, J. S. et al., Oligonucleotides, 14, 130-46, 2004) and ENA which is a nucleic acid derivative wherein the conformation of the sugar region is fixed to N type by crosslinking 2′ oxygen and 4′ carbon via an ethylene residue (Morita, K. et al., Nucleosides Nucleotides Nucleic Acids, 22, 1619-21, 2003) can also be used preferably.

miRNA can be prepared, for example, by obtaining the information of the base sequence of miRNA of interest from miRBase database and the like, and synthesizing, based thereon, by a commercially available DNA/RNA automatic synthesizer (Applied Biosystems, Beckman Instruments etc.). The above-mentioned miRNA containing various modifications can also be chemically synthesized by a method known per se. Commercially available miRNAs such as has-miR-148a-3p (Life Technologies Corporation) can also be utilized.

(b) siRNA to mRNA of Dnmt-1 Gene or a Precursor Thereof

In the present specification, double stranded RNA consisting of oligoRNA complementary to mRNA of Dnmt-1 gene and a complementary chain thereof, namely siRNA, is also defined to be encompassed in a nucleic acid containing a base sequence complementary or substantially complementary to the base sequence of mRNA of Dnmt-1 gene or a part thereof. When a short double stranded RNA is intracellularly introduced, mRNA complementary to RNA thereof is degraded, which is a phenomenon called RNA interference (RNAi) known for long in nematode, insect, plant and the like. Ever since this phenomenon was confirmed to widely occur in animal cells as well [Nature, 411(6836): 494-498 (2001)], it has been widely used as alternative technology for ribozyme.

siRNA to mRNA of Dnmt-1 gene is not particularly limited as long as it has RNAi activity for mRNA of Dnmt-1 gene, and can be designed based on the cDNA sequence information of the target gene and in accordance with, for example, the rules proposed by Elbashir et al. (Genes Dev., 15, 188-200 (2001)). Examples of the target sequence of siRNA include, but are not limited to, AA+(N)19, AA+(N)21 or NA+(N)21 (N is any base) and the like. Also, the position of the target sequence is not particularly limited. The selected candidate group of the target sequences is examined for the presence of homology with a sequence of 16-17 continuous bases in mRNA other than the target by using a homology search software such as BLAST (http://www.ncbi.nlm.nih.gov/BLAST/) and the like, and the specificity of the selected target sequence is confirmed. For example, when AA+(N)19, AA+(N)21 or NA+(N)21 (N is any base) is the target sequence, for the target sequence for which the specificity has been confirmed, a double stranded RNA consisting of a sense strand having a 3′-terminal overhang of TT or UU on 19-21 bases after AA (or NA), and an antisense strand having a sequence complementary to said 19-21 bases and a 3′-terminal overhang of TT or UU, is designed as RNA. In addition, short hairpin RNA (shRNA), which is a precursor of siRNA, can be designed by appropriately selecting any linker sequence (e.g., about 5-25 bases) capable of forming a loop structure and connecting the above-mentioned sense strand and antisense strand via the linker sequence.

siRNA and/or shRNA sequence can be searched for by using a homology search software provided free of charge on various websites. Examples of such site include, but are not limited to, siRNA Target Finder provided by Ambion (http://www.ambion.com/jp/techlib/misc/siRNA_finder.html) and insert design tool for pSilencer™ Expression Vector (http://www.ambion.com/jp/techlib/misc/psilencer_converter.html), GeneSeer provided by RNAi Codex (http://codex.cshl.edu/scripts/newsearchhairpin4.cgi).

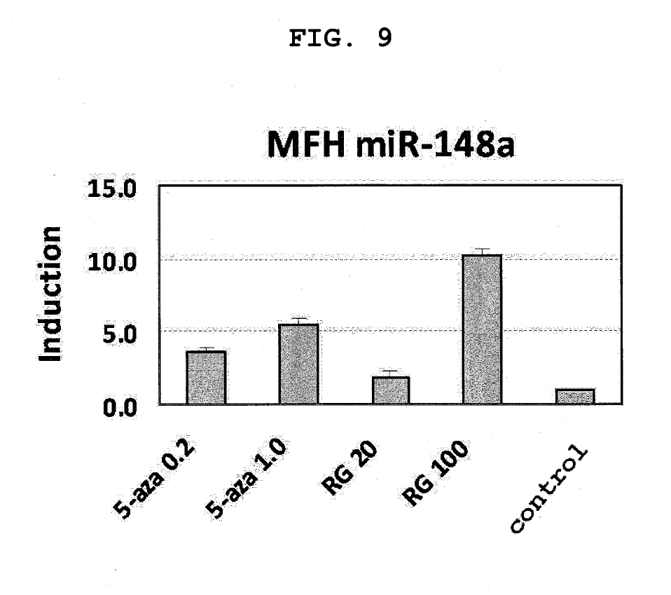

Examples of siRNA to mRNA of Dnmt-1 gene include, but are not limited to, commercially available siRNAs such as a double stranded RNA consisting of a sense strand composed of the base sequence shown in SEQ ID NO: 9 and an antisense strand shown in SEQ ID NO: 10 (siDnmt-la in FIG. 8) (Life Technologies Corporation), a double stranded RNA consisting of a sense strand composed of the base sequence shown in SEQ ID NO: 11 and the antisense strand shown in SEQ ID NO: 12 (siDnmt-1b in FIG. 8) (Life Technologies Corporation) and the like.

To improve stability, specific activity and the like, siRNA may be modified in the same manner as in the above-mentioned miRNA.

siRNA can be prepared by synthesizing each of the sense strand and antisense strand of the target sequence on mRNA by a DNA/RNA automatic synthesizer, denaturing them in a suitable annealing buffer at about 90-about 95° C. for about 1 min, and annealing them at about 30-about 70° C. for about 1-about 8 hrs. It can also be prepared by synthesizing shRNA to be a precursor of siRNA, and cutting same with a dicer.

siRNA can also be introduced into a cell as a precursor designed to be able to produce siRNA to mRNA of Dnmt-1 gene in vivo. As a precursor of siRNA, shRNA can be mentioned. shRNA can be prepared by designing an oligoRNA containing a base sequence formed by connecting a sense strand and an antisense strand of the target sequence on mRNA by inserting, between them, a spacer sequence having a length (e.g., about 5-25 bases) capable of forming a suitable loop structure, and synthesizing same by a DNA/RNA automatic synthesizer.

The above-mentioned “nucleic acid containing a base sequence complementary or substantially complementary to the base sequence of mRNA of Dnmt-1 gene or a part thereof” (hereinafter to be also referred to as the nucleic acid of the present invention) may be incorporated into a vector in the form of an expression vector. A vector expressing the nucleic acid of the present invention may be any as long as it is designed to biosynthesize the nucleic acid of the present invention such as miRNA and the like by transcription upon introduction into the cell. For example, the vector expressing siRNA or shRNA includes a tandem type and a stem-loop (hairpin) type. The former includes an expression cassette of the sense strand and an expression cassette of the antisense strand of siRNA connected in tandem, and forms double stranded siRNA (dsRNA) by intracellular expression and annealing of each chain. On the other hand, the latter is a shRNA expression cassette inserted into a vector and forms dsRNA by intracellular expression of shRNA and processing by dicer.

As promoters for such expression vectors, polII promoter (e.g., CMV early-immediate promoter) can be used. For accurate transcription of short RNA, polIII promoter is generally used. Examples of the polIII promoter include mouse and human U6-snRNA promoters, human H1-RNase P RNA promoter, human valine-tRNA promoter and the like. As a transcription termination signal, a sequence containing T continuously in the number of 4 or more is used. Specific examples of the vector capable of intracellularly expressing the nucleic acid of the present invention such as miRNA and the like include pCDNA6.2-GW/miR (manufactured by Invitrogen), pSilencer4.1-CMV (manufactured by Ambion), pSINsi-hH1 DNA (manufactured by Takara Bio Inc.), pSINsi-hU6 DNA (manufactured by Takara Bio Inc.), pENTR/U6 (manufactured by Invitrogen) and the like.

When the above-mentioned substance that suppresses expression of Dnmt-1 (preferably, the nucleic acid of the present invention) is introduced into a cell, lipofection method, liposome method, polyamine method, electroporation method, bead method and the like can be used as an introduction method. A method including forming a lipid complex with a cationic lipid and introducing same into a cell is most general and shows high introduction efficiency. For example, 0.1-100 μM, preferably 1-10 μM, of nucleic acid can be introduced into a mammalian cell using a commercially available nucleic acid introduction reagent. As an introduction reagent, TransFectin (Bio-Rad), Lipofectamine RNAiMAX (Invitrogen Corporation), HiPerFect Transfection Kit (QUIAGEN), DharmaFEC T reagent kit (Thermo Fisher Scientific) and the like can be used.

A vector expressing the nucleic acid of the present invention can be introduced into a cell by a method known per se according to the kind of the vector. In the case of a virus vector, for example, a plasmid containing a nucleic acid encoding a dominant-negative mutant is introduced into a suitable packaging cell (e.g., Plat-E cell) or a complementary cell line (e.g., 293 cell), a virus vector produced in the culture supernatant is recovered and the cell is infected with the vector by an appropriate method according to each virus vector. In the case of a plasmid vector which is a non-virus vector, the vector can be introduced into a cell by lipofection method, liposome method, electroporation method, calcium phosphate coprecipitation method, DEAE dextran method, microinjection method, particle gun method and the like.

In the present invention, “a substance that inhibits the function of Dnmt-1” may be any as long as an introduction of a methylation state of the parent chain into the daughter chain in the replication process of DNA due to the DNA methyltransferase activity by functionally produced Dnmt-1 is suppressed. Whether the function of Dnmt-1 is inhibited can be confirmed using the aforementioned in vivo or in vitro DNA methyltransferase activity measurement system of Dnmt-1 known per se. As a substance that inhibits the function of Dnmt-1, a substance that inhibits DNA methylation caused by Dnmt-1 can be mentioned. Preferable examples of the substance that inhibits DNA methylation cause by Dnmt-1 include a substance that is incorporated as a nucleotide analogue during DNA synthesis and inhibits methylation, and cytidine analogs such as 5-azacytidine, 5-aza-2′-deoxycytidine (decitabine), 1β-D-ribofuranosyl-2(1H)-pyrimidinone (zebularine) and the like, and a salt thereof, a solvate (e.g., ethanol solvate etc.) thereof, a hydrate thereof, a precursor or derivative (e.g., esterified derivative etc.) thereof can be mentioned. As the substance that inhibits DNA methylation caused by Dnmt-1, non-nucleotide compounds are also known, and preferable examples thereof include a substance that inhibits DNA methylation by masking a Dnmt-1 target sequence (e.g., procaine, procainamide etc.), and a substance that inhibits DNA methylation by blocking the active site of Dnmt-1 (e.g., ((−)-epigallocatechin-3-gallate (EGCG), RG108 etc.). Of the above-mentioned substances that inhibit DNA methylation, RG108 is preferable since it shows a high effect of promoting differentiation into a hepatocyte.

In addition, a substance that inhibits the function of Dnmt-1 may be a substance that inhibits, whether reversibly or irreversibly, the intracellular function of Dnmt-1 by directly or indirectly acting on Dnmt-1. Examples of such substance include a substance that inhibits the intracellular function of Dnmt-1 by inhibiting formation of a complex of Dnmt-1 and other protein (e.g., histone deacetylases (HDAC) etc.), a substance that promotes degradation of Dnmt-1 (e.g., a substance that promotes degradation of Dnmt-1 via proteasome (e.g., 5-aza-2′-deoxycytidine etc.)) and the like.

To be specific, as a substance that inhibits the function of Dnmt-1, for example, a neutralization antibody to Dnmt-1 (i.e., antibody that inhibits the function of Dnmt-1 by binding to Dnmt-1 (e.g., antibody that inhibits binding between Dnmt-1 and HDAC)) can be mentioned. The antibody may be any of a polyclonal antibody and a monoclonal antibody. These antibodies can be selected by screening anti-Dnmt-1 antibodies produced according to the production methods known per se of antibody or anti-serum, for whether or not they inhibit the function of Dnmt-1. While the isotype of antibody is not particularly limited, it is preferably IgG, IgM or IgA, particularly preferably IgG. The antibody is not particularly limited as long as it has at least a complementarity determination region (CDR) for specifically recognize and bind to the target antigen, and it may be a complete antibody molecule or, for example, fragments of Fab, Fab′, F(ab′)2 and the like, conjugate molecules produced by genetic engineering such as scFv, scFv-Fc, minibody, diabody and the like, and derivatives thereof which are modified by a molecule having a protein-stabilizing action such as polyethylene glycol (PEG) and the like, and the like.

When the “substance that inhibits the function of Dnmt-1” is a polypeptide, the polypeptide can be introduced into a cell by a method known per se for introducing a protein into a cell. Examples of such method include, but are not limited to, a method using a protein introduction reagent, a method using a protein introduction domain (PTD) or cell permeability peptide (CPP) fusion protein, a microinjection method and the like.

2. Method of Producing Hepatocyte from Hepatic Progenitor Cell or Hepatoblastoma

The present invention provides a method of producing a hepatocyte from a hepatic progenitor cell or hepatoblastoma, comprising the following steps (hereinafter to be also referred to as the method of the present invention):

(1) a step of suppressing expression of Dnmt-1 or inhibiting function of Dnmt-1 in a hepatic progenitor cell or hepatoblastoma by contacting the hepatic progenitor cell or hepatoblastoma with the promoter of the present invention (i.e., a substance that suppresses expression of Dnmt-1 or a substance that inhibits the function of Dnmt-1); and
(2) a step of inducing differentiation into a hepatocyte by culturing the hepatic progenitor cell or hepatoblastoma obtained in the aforementioned step (1), wherein expression of Dnmt-1 is suppressed or function of Dnmt-1 is inhibited.

The method of the present invention is based on the promotion of differentiation from hepatic progenitor cell or hepatoblastoma into hepatocyte by the promoter of the present invention, and can produce hepatocyte efficiently. Hepatocyte is a cell as described in the above-mentioned 1. In a preferable embodiment, the hepatocyte produced by the method of the present invention is a hepatocyte with high maturity. In the present invention, the “hepatocyte with high maturity” means a hepatocyte having a high expression level for at least one kind of hepatocyte markers (e.g., albumin, glucose-6-phosphatase (G6pc), tyrosine aminotransferase (Tat), cytochrome P450 (Cyp), miR-122 etc.) (preferably, 4 kinds of albumin, G6pc, Tat and Cyp, more preferably 5 kinds of albumin, G6pc, Tat, Cyp and miR-122), as compared to hepatocytes produced by a method similar to the method of the present invention except that a hepatic progenitor cell or hepatoblastoma without introduction of the promoter of the present invention is used. Preferably, the expression level of a hepatocyte marker is the same as or not less than that of primary culture hepatocyte. The expression level of a hepatocyte marker can be measured by a method known per se, as described in the above-mentioned 1.

The hepatic progenitor cell or hepatoblastoma in step (1) is as described in the above-mentioned 1. The hepatic progenitor cell or hepatoblastoma used in the present invention is generally isolated. In the present invention, being “isolated” means being intentionally placed in a state different from naturally-occurring states. For example, primary cultured cells collected from a living organism and cells induced to differentiate from stem cells are isolated cells.

The contact between a hepatic progenitor cell or hepatoblastoma and the promoter of the present invention (i.e., a substance that suppresses expression of Dnmt-1 or a substance that inhibits the function of Dnmt-1) in step (1) can be performed by, as described in the above-mentioned 1, introducing the substance into a hepatic progenitor cell or hepatoblastoma. When the substance is a nucleic acid, from the aspect of efficiency of introduction into cells, it is preferable to form a lipid complex with a cationic lipid and introduce same into a hepatic progenitor cell or hepatoblastoma, which can be performed, for example, by the following method: using a commercially available nucleic acid introduction reagent (e.g., TransFectin (Bio-Rad) etc.), a lipid complex of the promoter of the present invention and a cationic lipid is formed according to the description in the attached instruction manual. After culture of hepatic progenitor cell or hepatoblastoma for a given time (e.g., about 1 day, 2 days, 3 days, 4 days), the complex is added to a medium to allow contact with the hepatic progenitor cell or hepatoblastoma, and the mixture is further cultured for a given time (e.g., about 5 hrs) to introduce the promoter of the present invention into the hepatic progenitor cell or hepatoblastoma. For culture of the hepatic progenitor cell or hepatoblastoma, a medium widely used for culturing animal cells can be utilized as a basal medium. A commercially available basal medium may also be utilized. Examples thereof include, but are not particularly limited, (minimum essential medium (MEM), Dulbecco's modified minimum essential medium (DMEM), RPMI1640 medium, 199 medium, William's E medium) and the like. These can be used singly, or two or more kinds thereof can be used in combination. Examples of the additive to a medium include various amino acids (e.g., L-glutamine etc.), various inorganic salts, various vitamin, various antibiotics (e.g., penicillin, streptomycin etc.), buffering agents and the like. Generally, introduction of a nucleic acid such as miRNA, siRNA and the like into a cell is performed when the cell is in a logarithmic growth phase (e.g., when cells cover 80% of the bottom surface of the culture container (80% confluent)), it is preferable to add a serum (e.g., fetal bovine serum etc.) to the medium to promote growth of hepatic progenitor cells. Generally, moreover, factors such as growth factor, cytokine, hormone and the like are also added. Examples of the factor include epidermal growth factor (EGF) (about 25 ng/mL), insulin (about 5 μg/mL), hepatocyte growth factor (HGF) (about 25 ng/mL), oncostatin M (about 12.5 ng/mL), hydrocortisone 21-hemisuccinic acid or a salt thereof (about 5×10⁻⁷ M) and dexamethasone (about 10⁻⁷ M). Particularly, EGF is preferably added to the medium to promote growth of hepatic progenitor cells. In a further aspect, a serum (e.g., fetal bovine serum etc.) is preferably added to a medium for growing hepatoblastoma to promote the growth of hepatoblastoma.

When a hepatic progenitor cell or hepatoblastoma is contacted with the promoter of the present invention, it is preferable, from the aspect of introduction efficiency, to exchange a medium suitable for the growth of hepatic progenitor cell or hepatoblastoma as mentioned above (to be also referred to as a medium for hepatic progenitor cell or hepatoblastoma in the present specification) with a special medium used for transfection (also referred to as a medium for transfection) such as OptiMEM (Invitrogen Corporation) and the like.

Since hepatocyte can be obtained efficiently by inducing differentiation into hepatocyte after reaching confluence of hepatic progenitor cell or hepatoblastoma, when the number of cells is small after culture in the aforementioned medium for transfection, the cells may be further cultured in a medium for hepatic progenitor cell or hepatoblastoma. Such additional culture in the medium for hepatic progenitor cell or hepatoblastoma may be performed until the cells reach confluence. Generally, the total culture time in the medium for hepatic progenitor cell or hepatoblastoma, including the culture time in the medium for hepatic progenitor cell or hepatoblastoma before contact with the promoter of the present invention, is not less than 3 days (about 4 days).

In addition, the contact between a hepatic progenitor cell or hepatoblastoma and the promoter of the present invention (i.e., a substance that suppresses expression of Dnmt-1 or a substance that inhibits the function of Dnmt-1) can be performed by culturing hepatic progenitor cell or hepatoblastoma in the presence of the promoter of the present invention. Examples of the medium include the above-mentioned medium used for introducing the promoter of the present invention into a hepatic progenitor cell or hepatoblastoma, and a medium for hepatic progenitor cell or hepatoblastoma. While the contact conditions are appropriately determined, for example, pH is about 6-about 8, and the temperature is generally about 30-about 40° C. The contact time and the concentration of the promoter of the present invention on contact are not particularly limited as long as expression of Dnmt-1 is suppressed or the function of Dnmt-1 is inhibited in the hepatic progenitor cell or hepatoblastoma, and can be appropriately determined according to the active ingredient of the promoter of the present invention. For example, when 5-aza-2′-deoxycytidine (decitabine) is used as the promoter of the present invention, hepatic progenitor cells are cultured in the medium for hepatic progenitor cell, and the promoter of the present invention at a concentration of about 0.2-1.0 μM is brought into contact therewith from day 2 of culture until the cells become confluent. In a further aspect, they may be contacted with hepatoblastoma from day 0 of culture until the cells become confluent in the medium for hepatoblastoma added with 5-aza-2′-deoxycytidine (decitabine) at a concentration of 2.5-20 μM. For example, when RG108 is used as the promoter of the present invention, hepatic progenitor cells are cultured in the medium for hepatic progenitor cell, and the promoter of the present invention at a concentration of about 20-100 μM is brought into contact therewith from day 2 of culture until the cells become confluent. In a further aspect, they may be contacted with hepatoblastoma from day 0 of culture until the cells become confluent in the medium for hepatoblastoma added with RG108 at a concentration of about 20-100 μM. When hepatic progenitor cell or hepatoblastoma is contacted with the promoter of the present invention by culturing hepatic progenitor cell or hepatoblastoma in the presence of the promoter of the present invention, the culture in the below-mentioned step (2) is also preferably performed in the presence of the promoter of the present invention to more efficiently promote differentiation into a hepatocyte. In this case, the concentration of the promoter of the present invention only needs to be of the same level as in step (1).

The culture in step (2) can be performed by any method as long as induction of differentiation from hepatic progenitor cell into hepatocyte is possible, and a known method can be used as a method for differentiation induction into hepatocyte. Examples of the known method include a method of culturing in a culture medium added with oncostatin M, dexamethasone, hepatocyte growth factor (HGF) and the like (Journal of Cellular Physiology, Vol. 227(5), p. 2051-2058 (2012); Hepatology, Vol. 45(5), p. 1229-1239 (2007)) and the like. As a differentiation induction method from hepatoblastoma into hepatocyte, a method of culturing continuously in a culture medium and the like can be mentioned.

For culture in step (2), a medium widely used for culturing animal cells can be utilized as a basal medium (also referred to as basal medium for hepatocyte in the present specification). A commercially available basal medium may also be utilized. Examples thereof include, but are not particularly limited, (minimum essential medium (MEM), Dulbecco's modified minimum essential medium (DMEM), RPMI1640 medium, 199 medium, William's E medium) and the like. These can be used singly, or two or more kinds thereof can be used in combination. Examples of the additive to a medium include various amino acids (e.g., L-glutamine etc.), various inorganic salts, various vitamin, various antibiotics (e.g., penicillin, streptomycin etc.), buffering agents and the like.

For differentiation induction from hepatic progenitor cell into hepatocyte, generally, factors such as growth factor, cytokine, hormone and the like are also added to the above-mentioned basal medium for hepatocyte. As long as a basal medium for hepatocyte added with these factors (also referred to as differentiation induction medium in the present specification) can induce differentiation from hepatic progenitor cell into hepatocyte, the factors to be added are not particularly limited. For example, as described in the below-mentioned Examples, differentiation induction from hepatic progenitor cell into hepatocyte can be performed by using a medium added with, as these factors, insulin (about 5 μg/mL), hepatocyte growth factor (HGF) (about 25 ng/mL), oncostatin M (about 12.5 ng/mL), hydrocortisone 21-hemisuccinic acid or a salt thereof (about 5×10⁻⁷ M) and dexamethasone (about 10⁻⁷ M). For example, as disclosed in JP-A-2000-287680, differentiation induction from immature hepatocyte into hepatocyte is also possible by using oncostatin M and dexamethasone from among the above-mentioned factors. To grow hepatic progenitor cells, a medium added with EGF and serum (e.g., fetal bovine serum etc.) is used; however, use of a medium free of these is preferable for differentiation induction.

For differentiation induction from hepatoblastoma into hepatocyte, a serum (e.g., fetal bovine serum etc.) is generally further added to the above-mentioned basal medium. As long as a medium for hepatoblastoma added with these factors (also referred to as medium for hepatoblastoma in the present specification) can induce differentiation from hepatoblastoma into hepatocyte, the factors to be added are not particularly limited.

In one embodiment, regarding EGF and serum in the above-mentioned basal medium for hepatocyte (differentiation induction medium), a medium free of EGF and having a decreased serum concentration can also be used. The amount of the reduced serum may be 0.5-2%(w/w), preferably 1%(w/w).

Differentiation of hepatic progenitor cell or hepatoblastoma obtained in step (1) wherein the expression of Dnmt-1 is suppressed or the function of Dnmt-1 is inhibited can be induced by culturing same in a differentiation induction medium or a medium for hepatoblast. The culture conditions are not particularly limited as long as differentiation from hepatic progenitor cell or hepatoblastoma into hepatocyte is induced and are appropriately determined. For example, the pH of the medium is about 6-about 8 and the culture temperature is generally about 30-about 40° C. Also, the culture time is not particularly limited, and can be appropriately determined according to the method of differentiation induction into hepatocyte.

In the above-mentioned steps (1) and (2), differentiation from hepatic progenitor cell or hepatoblastoma into hepatocyte is promoted and the hepatocyte can be produced efficiently. Production of hepatocyte can be confirmed using expression of a hepatocyte marker as an index. The expression level of a hepatocyte marker can be measured by a method known per se, as described in the above-mentioned 1. Hepatocyte, generally has functions such as glucose production capacity, ammonia metabolic capacity, albumin production capacity, urea synthesis ability and the like. Therefore, production of hepatocyte can also be confirmed using the presence or absence of these functions as an index. The glucose production capacity can be confirmed by analyzing the glucose level of culture supernatant by a glucose oxidase method. The ammonia metabolic capacity can be confirmed by analyzing the ammonia level of culture supernatant by a modified indophenol method (Horn D B & Squire C R, Clin. Chim. Acta. 14(2): p. 185-94 (1966)). The albumin production capacity can be confirmed by analyzing the albumin concentration of culture supernatant by a method of measuring the serum albumin concentration. In addition, the urea synthesis ability can be confirmed, for example, by using colorimetric assay (Sigma Ltd.). In a preferable embodiment of the present invention, production of hepatocyte having high maturity can be confirmed by measuring the expression level of a hepatocyte marker and comparing said level with that of control (expression level when differentiation induction of hepatic progenitor cell or hepatoblastoma without introduction of the promoter of the present invention is performed in the same manner).

According to the method of the present invention, hepatocyte used for the prediction of hepatotoxicity and liver metabolite, and the like can be supplied stably. In a preferable embodiment of the present invention, since a hepatocyte with high maturity as compared to hepatocytes produced by a conventional method can be produced, hepatocyte more preferably used for the prediction of hepatotoxicity and liver metabolite, and the like can be supplied stably. Therefore, the method of the present invention is useful in the fields of, for example, development of pharmaceutical products, food and the like.

When a substance having an activity to suppress expression of Dnmt-1, or inhibit the function of Dnmt-1 activity is added to a medium for differentiation induction of hepatocyte, the substance may show cytotoxicity. Therefore, when differentiation into hepatocyte is induced using the promoter of the present invention, the cytotoxicity can be reduced by seeding at high cell density. Preferable cell density at seeding is, for example, at 4×10⁴-2×10⁵ cells/cm². Particularly, when differentiation induction is performed for a long term, seeding at the above-mentioned cell density is preferable to suppress cytotoxicity of the substance having an activity to suppress expression of Dnmt-1, or inhibit the function of Dnmt-1.

In a preferable embodiment of the present invention, the culture in the above-mentioned step (2) is performed for a long term. A long-term differentiation induction affords hepatocyte with more advanced maturity. In such cell, an increase in the expression of a hepatocyte marker is induced in not only the mRNA level but also protein level. A preferable period of long-term differentiation induction is not particularly limited as long as the expression of a hepatocyte marker at a protein level is induced. For example, not less than 7 days, not less than 10 days, not less than 13 days and the like can be mentioned.

To confirm expression induction of hepatocyte marker at a protein level in hepatocyte with more advanced maturity, the protein amount of the above-mentioned hepatocyte marker can be quantified by a method known per se such as Western blotting and the like, or for example, the activity of CYP enzymes (e.g., CYP1A2 and CYP3A4 etc.) can also be measured.

Alternatively, hepatocyte with more advanced maturity has a specific morphology with multiple nuclei, clear cell boundary under a microscope and the like, and/or may show promoted glycogen storage capacity. Whether the cell has multiple nuclei can be determined by a confirmation method using an optical microscope, a method of staining the nucleus with a known nuclear staining agent (e.g., DAPI, Hoechst33342 etc.) and the like, and clear cell boundary can be confirmed by an optical microscope. In addition, glycogen storage capacity can be confirmed by a method known to those of ordinary skill in the art such as PAS staining and the like. In a preferable embodiment, hepatocyte with advanced maturity, which is induced by long-term culture, expresses CYP1A2 and CYP3A4 proteins, has multiple nuclei and PAS staining positive.

The present invention also provides hepatocyte produced by the method of the present invention. The hepatocyte produced by the method of the present invention can be utilized, for example, for the evaluation of metabolism and hepatotoxicity of a test compound and the like.

For the evaluation of metabolism and hepatotoxicity of a test compound, animal model and the like have conventionally been used. However, the number of test compounds that can be evaluated at one time is limited, and evaluation obtained in animal model and the like cannot be directly applied to human. Therefore, an evaluation method using human liver cancer cell line and primary no/mal human cultured hepatocyte has been adopted. However, since human liver cancer cell line is a cancer cell, the evaluation obtained in human liver cancer cell line may not be applicable to human normal hepatocyte. In addition, primary normal human cultured hepatocyte poses problems in terms of stable supply and cost. Moreover, a cell line immortalizing primary normal human cultured hepatocyte shows decreased CYP3A4 activity as compared to the absence of immortalization (International Journal of Molecular Medicine 14: 663-668, 2004, Akiyama I. et al.). Such problems can be solved by utilizing hepatocyte produced by the method of the present invention.

Accordingly, the present invention further provides a method of evaluating the metabolism of a test compound. In the method, hepatocyte produced by the method of the present invention is contacted with a test compound. Then, the metabolism of the test compound contacted with the hepatocyte is measured.

The test compound to be used in the present invention is not particularly limited. Examples thereof include, but are not limited to, single compounds such as xenobiotic substance, natural compound, organic compound, inorganic compound, protein, peptide and the like, and expression products of compound library and gene library, cell extract, cell culture supernatant, fermentation microorganism product, marine organism extract, plant extract and the like.

Examples of the xenobiotic substance include, but are not limited to, candidate compounds of medicament and food, existing medicament and food, and any substance foreign to living organisms is included in the xenobiotic substance in the present invention. More specifically, Rifampin, Dexamethasone, Phenobarbital, Ciglirazone, Phenytoin, Efavirenz, Simvastatin, β-Naphthoflavone, Omeprazoie, Clotrimazole, 3-Methylcholanthrene and the like can be recited as examples.

The “contact” in the present invention is generally made by adding a test compound to a medium or a culture medium, but the method is not limited thereto. When the test compound is a protein and the like, the “contact” can be made by introducing a DNA vector expressing the protein into the cell.

The metabolism of a test compound can be measured by a method well known to those of ordinary skill in the art. For example, when a metabolite of the test compound is detected, the test compound is judged to have been metabolized. In addition, when the expression of enzyme genes such as CYP (cytochrome p450), MDR, MPR and the like is induced, or the activity of these enzymes increased by contact with the test compound, the test compound is judged to have been metabolized.

Also, the present invention provides an evaluation method of hepatotoxicity of a test compound. In the method, hepatocyte produced by the method of the present invention is contacted with a test compound. Then, the level of disorder in the hepatocyte contacted with the test compound is measured. The level of disorder can be measured, for example, by using a survival rate of the hepatocyte or hepatopathy markers such as GOT, GPT and the like as an index.

For example, when the survival rate of hepatocyte decreases by the addition of a test compound to the culture medium of the hepatocyte, the test compound is judged to have hepatotoxicity, and when the survival rate does not change significantly, the test compound is judged to have no hepatotoxicity. For example, when GOT and GPT in the culture medium of hepatocyte increase after addition of a test compound, the test compound is judged to have hepatotoxicity, and when GOT and GPT do not show a significant change, the test compound is judged to have no hepatotoxicity.

When a compound having determined hepatotoxicity is used as a control, the presence or absence of hepatotoxicity in the test compound can be evaluated more accurately.

### EXAMPLES

The Examples of the present invention are explained in the following, which are not to be construed as limitative.

### Materials and Methods

### (1) Recovery of Hepatic Tissue

### (i) Preparation of HEPES Buffer

To autoclave-sterilized distilled water (750 mL) were added 8 g of sodium chloride (Sigma-Aldrich Corporation), 0.2 g of potassium chloride (Sigma-Aldrich Corporation), 0.1 g of sodium hydrogenphosphate dodecahydrate (Wako Pure Chemical Industries, Ltd.) and 10 mL of HEPES solution (2.38 g/10 mL, Invitrogen Corporation), and the mixture was adjusted to 1 L with stirring. To the obtained HEPES buffer (100 mL) was added 56.25 mg of calcium chloride dihydrate (Sigma-Aldrich Corporation) to prepare 100 mL of HEPES buffer (containing calcium).

### (ii) Recovery of Mouse Embryo-Derived Hepatic Tissue

Two pregnant mice (E14.5) were anesthetized and sacrificed by cervical dislocation. Abdominal cavity was opened with surgical scissors, and uterus containing embryo was carefully removed and transferred into ice-cooled PBS solution (Sigma-Aldrich Corporation: D8537). The embryo was removed with tweezers and transferred into a 10 cm petri dish containing HEPES buffer. Using stereoscopic microscope, the embryo was anatomize and hepatic tissue was recovered. The recovered hepatic tissue was transferred into the petri dish containing HEPES buffer, hepatic capsule was carefully removed to give a hepatic tissue, which was used for collection of hepatic parenchymal cells.

### (2) Collection of Hepatic Parenchymal Cell

### (i) Preparation of Enzyme Solution

To 5 mg of freeze-dried liberase (Roche) was added 2 mL of distilled water, and the mixture was left standing in ice for 30 min, and stirred every 2-3 minutes until complete dissolution to prepare 13 U/mL of liberase solution. The 13 U/ml of liberase solution was used as a stock solution and preserved at −20° C. When in use, 100 μL of liberase solution (13 U/mL) was added to 10 mL of HEPES buffer (containing calcium) to prepare an enzyme solution.

### (ii) Preparation of Basal Medium for Hepatocyte

To William's E medium (Invitrogen Corporation) were added 2 mM of L-glutamine solution (Invitrogen Corporation), 50 U/mL of penicillin-50 μg/mL of streptomycin solution (Invitrogen Corporation) and 10% fetal bovine serum (Thermo Fisher Scientific K.K.) to prepare the medium.

(iii) Collection of Embryo Hepatic Tissue-Derived Hepatic Parenchymal Cells

The recovered hepatic tissue was transferred into a 1.5 mL tube, and thoroughly cut into small pieces with surgical scissors. An enzyme treatment was performed by the following procedures. The minced tissue was placed in a 50 mL tube, suspended in 10 mL of enzyme solution warmed to 37° C., and incubated at 37° C. for 5 min, during which the tube was stirred every 2-3 minutes. Thereafter, 25 mL of the basal medium for hepatocyte was added to a 2 mL plastic pipette, and the enzyme reaction was quenched by diluting the enzyme solution. The diluted enzyme solution containing the cells was filtered with a 60 μm nylon filter to give a cell suspension. Subsequently, the filter was washed with the basal medium for hepatocyte, and the medium was recovered in the same container as the cell suspension. The cell suspension was adjusted to about 100 mL. The cell suspension was transferred into Erlenmeyer flask, and left standing at room temperature for 15 min. After standing, the upper layer (three-quarters of total volume) was removed with a 25 mL plastic pipette. To the remaining cell suspension was added 25 mL of the basal medium for hepatocyte, and the mixture was suspended again. The suspension was divided into two 50 mL round-bottom tubes with a 25 mL plastic pipette, the basal medium for hepatocyte was added to each tube, and the volume was adjusted to 40 mL. The mixture was centrifuged at 4° C., 1000 rpm (22×g) for 2 min, and the supernatant was removed to give hepatic parenchymal cells.

### (3) Isolation of Hepatic Progenitor Cell

### (i) Preparation of Medium for Hepatic Progenitor Cell

To a basal medium for hepatocyte were added 5 μg/mL of insulin (Sigma-Aldrich Corporation), 25 ng/mL of epidermal growth factor (EGF) (Sigma-Aldrich Corporation), 25 ng/mL of hepatocyte growth factor (HGF) (Peprotech), 12.5 ng/mL of oncostatin M (Sigma-Aldrich Corporation), 5×10⁻⁷M hydrocortisone 21-hemisuccinate sodium salt (Sigma-Aldrich Corporation) and 10⁻⁷ M dexamethasone (Sigma-Aldrich Corporation) to prepare the medium.

### (ii) Isolation of Hepatic Progenitor Cell

The hepatic parenchymal cells were diluted with HEPES buffer added with 2% fetal bovine serum to prepare a 10⁷ cells/100 μL cell suspension, which was transferred into a 5 mL round-bottom tube. A mouse FcR blocking antibody in EasySep Mouse Biotin Positive Selection Kit (STEMCELL Technologies Inc) was added at 1 μL per 10⁷ cells, and the mixture was admixed by tapping the tube. Biotin-labeled anti-E-cadherin antibody (eBioscience) was added at 2-4 μL per 10⁷ cells, and the mixture was left standing at room temperature for 15 min while stirring the tube every 2-3 minutes. Biotin Selection Cocktail in EasySep Mouse Biotin Positive Selection Kit was added at 10 μL per 10⁷ cells, and the mixture was left standing at room temperature for 15 min with occasional stirring. Magnetic particles in EasySep Mouse Biotin Positive Selection Kit were added at 5 μL per 10⁷ cells, and the mixture was stirred and left standing at room temperature for 10 min. To the cell suspension was added a medium for cell isolation, and the volume was adjusted to 2.5 mL. The mixture was suspended with a 2 mL plastic pipette, and the tube was placed in an exclusive magnet (STEMCELL Technologies Inc.) and left standing at room temperature for 10 min. The exclusive magnet containing the tube was reversed for 3 sec, and a non-hepatic progenitor cell fraction was removed (purification operation: first time). The tube was taken out from the exclusive magnet, 2.5 mL of the medium for cell isolation was added, and a purification operation was performed again (purification operation: second time). Furthermore, 2.5 mL of the medium for cell isolation was added, and a purification operation was performed for the third time (purification operation: third time). The cells remaining in the tube are the hepatic progenitor cells.

### (4) Culture of Liver Cancer Cell Line

Mouse liver cancer-derived cell line (Hepa1-6) and human liver cancer-derived cell lines (Huh-7, HepG2, Hep3B) were cultured in DMEM (Invitrogen Corporation) added with 50 U/mL penicillin-50 μg/mL streptomycin solution (Invitrogen Corporation) and 10% fetal bovine serum (Thermo Fisher Scientific K.K.).

### (5) Extraction of Total RNA

Total RNA was extracted from cultured cells, liver cancer cell lines, and mouse and human normal hepatic tissues by using miRNeasy Mini Kit (QUIAGEN). The total RNA was quantified by NanoDrop 1000 spectrophotometer (Thermo Fisher Scientific K.K.). The hepatic tissues recovered from mouse were placed in ice-cooled phosphate buffer and disrupted before extraction of RNA. Red blood cells were lysed in Red Blood Cell Lysis Buffer (Sigma-Aldrich Corporation).

### (6) Quantification by the Real-Time PCR Method

RNA was quantified by the real-time PCR method using 7300 real-time PCR system (Applied Biosystems) and TaqMan MicroRNA Assays (Applied Biosystems). The miRNA expression was standardized at 6NU6B level. For quantification of messenger RNA, total RNA sample was treated with DNase (TURBO DNA-free kit: Ambion Inc.), and cDNA was synthesized from 1 μg of total RNA by using reverse transcriptase (SuperScript III Reverse Transcriptase: Ambion Inc.). For quantification, SYBR Green (PlatinumSYBR Green quantitative PCR SuperMix UDG: Invitrogen Corporation) was used. The reaction for real-time PCR was performed at 95° C., 2 min, 40 cycles (95° C. for 15 sec, 60° C. for 30 sec). The cDNA expression level was standardized by GAPDH.

(7) Introduction of miRNA Precursor or siRNA into Cell

For introduction of miRNA precursor into liver cancer cell line, the cells were plated on a 35 mm dish for cell culture at 4×10⁵ cells and, after 24 hr of culture, 100 nM miRNA precursor and 5 μL of a transfectin reagent (Bio-Rad) were added to 1 mL of OptiMEM (Invitrogen Corporation). miRNA precursor was introduced into hepatic progenitor cells on day 4 of culture by a method similar to that of miRNA precursor into liver cancer cell line. siRNA was introduced into hepatic progenitor cell on day 2 of culture by a method similar to that of miRNA precursor into liver cancer cell line and using 100 nM siRNA (two kinds).

### (8) Culture of Human Hepatic Progenitor Cell

Human hepatic progenitor cells (Cell Systems Corporation) were cultured in DMEM (Invitrogen Corporation) added with 10% fetal bovine serum (MP Bio).

### (9) Extraction of Total RNA

Total RNA was extracted from human hepatic progenitor cell by using miRNeasy Mini Kit (QUIAGEN). Total RNA was quantified by Synergy H1-Take3 plate (BIOTEC Co., Ltd.).

### (10) Quantification by the Real-Time PCR Method

RNA was quantified by the real-time PCR method and using StepOne real-time PCR system (Applied Biosystems) and TaqMan Gene Expression Assays (Applied Biosystems). For quantification of messenger RNA, cDNA was synthesized from 1 μg of total RNA by using reverse transcriptase (ReverTra Ace qPCR RT Master Mix: TOYOBO CO., LTD.). For quantification, TaqMan Universal PCR Master Mix, No AmpErase UNG (Invitrogen Corporation) was used. The reaction for real-time PCR was performed at 50° C., 2 min and 95° C., 10 min, 40 cycles (95° C. for 15 sec, 60° C. for 1 min). The cDNA expression level was standardized by 18S ribosomal RNA.

(11) Introduction of miRNA Precursor into Human Hepatic Progenitor Cell

For introduction of miRNA precursor into human hepatic progenitor cell, the cells were plated on a 6-well plate at 2×10⁴ cells/cm² and, at 80% confluent, 20 nM miRNA precursor and 3.7 μL of a transfectin reagent (Bio-Rad) were added to 1.25 mL of OptiMEM (Invitrogen Corporation).

### Example 1

### Differentiation and Maturation of Hepatic Progenitor Cell

Mouse hepatic progenitor cells were plated at 2×10⁶ cells/cm² on a 35 mm cell culture dish coated with type I collagen, and cultured in a medium for hepatic progenitor cell. After confluence, the cells were cultured in a medium for hepatic progenitor cell free of epidermal growth factor (EGF) and fetal bovine serum. The medium was exchanged every day.

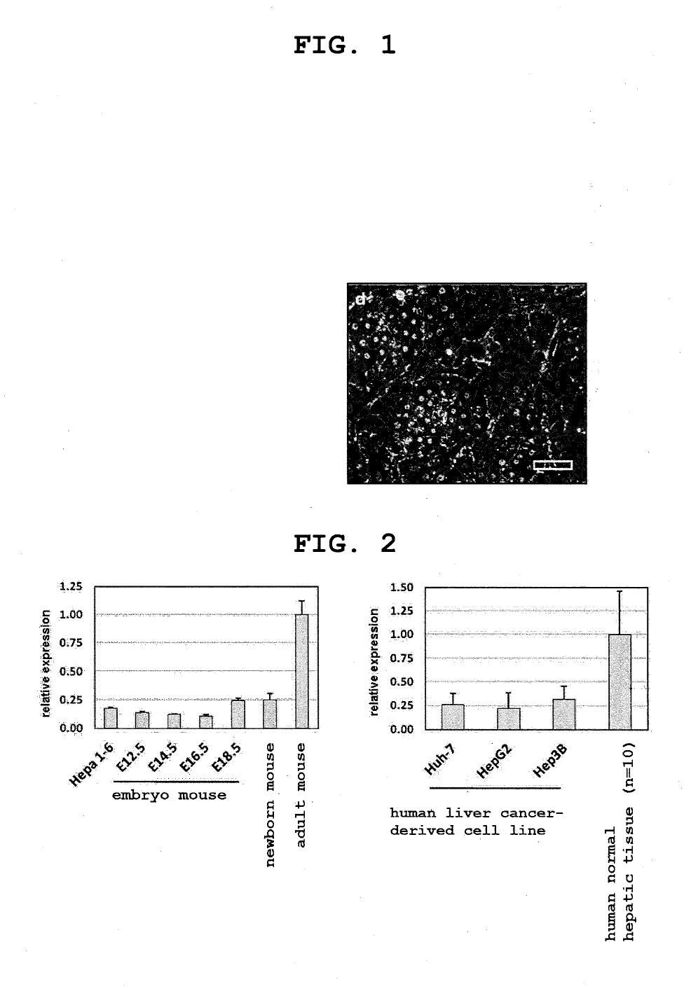

The morphological changes of the cells are shown in FIG. 1. The cells became confluent in 3 days of culture and the morphology of the cells was quadrate (FIG. 1a). The cell morphology became oval after 5 days of culture (FIG. 1b). On day 12 of culture, the cells showed morphology of matured hepatocyte (FIGS. 1c and d).

### Example 2

### Quantification of miR-148a in Human and Mouse Hepatic Tissues or Liver Cancer-Derived Cells

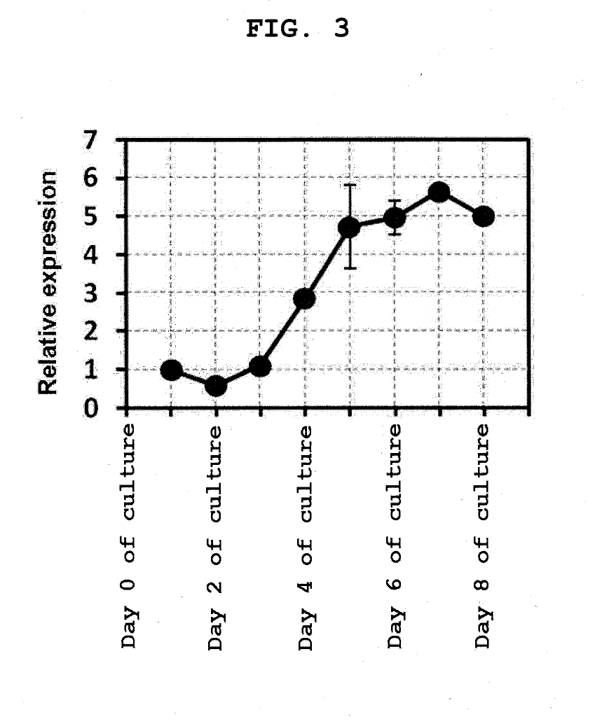

miR-148a expressed in mouse liver cancer-derived cell line Hepa1-6 (American Type Culture Collection: ATCC), human liver cancer-derived cell lines HepG2, Hep3B (American Type Culture Collection: ATCC), human liver cancer-derived cell line Huh-7 (RIKEN cell bank), mouse (embryo mouse, newborn mouse, adult mouse) hepatic tissues (collected by a method similar to that in the above-mentioned (1) and (2)) and human normal hepatic tissue (obtained from National Cancer Center) was quantified by the real-time PCR method. As shown in FIG. 2, miR-148a was highly expressed only in adult mouse hepatic tissue and human normal hepatic tissue.

### Example 3

### Changes in miR-148a Expression Level after Differentiation Induction of Hepatic Progenitor Cell

Mouse hepatic progenitor cells were plated at 4×10⁴ cells/cm² in a 35 mm cell culture dish coated with type I collagen, and cultured in a medium for hepatic progenitor cells. After confluence (after day 4 of culture), the cells were cultured in the medium for hepatic progenitor cells free of epidermal growth factor (EGF) and fetal bovine serum. The medium was exchanged every day. Total RNA containing miRNA was extracted, and miR-148a was quantified by the real-time PCR method.

As shown in FIG. 3, the miR-148a expression level increased with maturation of the hepatic progenitor cells after differentiation induction.

### Example 4

### Changes in miR-148a Expression Level after Differentiation Induction of Hepatic Progenitor Cell Introduced with miR-148a Precursor

Mouse hepatic progenitor cells were plated at 4×10⁴ cells/cm² in a 35 mm cell culture dish coated with type I collagen, and cultured in a medium for hepatic progenitor cells. miR-148a precursor was introduced into the cultured mouse hepatic progenitor cells and, after confluence (after day 4 of culture), the cells were cultured in the medium for hepatic progenitor cells free of epidermal growth factor (EGF) and fetal bovine serum. The medium was exchanged every day. Total RNA containing miRNA was extracted, and miR-148a was quantified by the real-time PCR method. When miR-148a precursor was not introduced, the measurement was started from day 2 of culture; when miR-148a precursor was introduced, the measurement was started from day 5 of culture after introduction of miRNA.

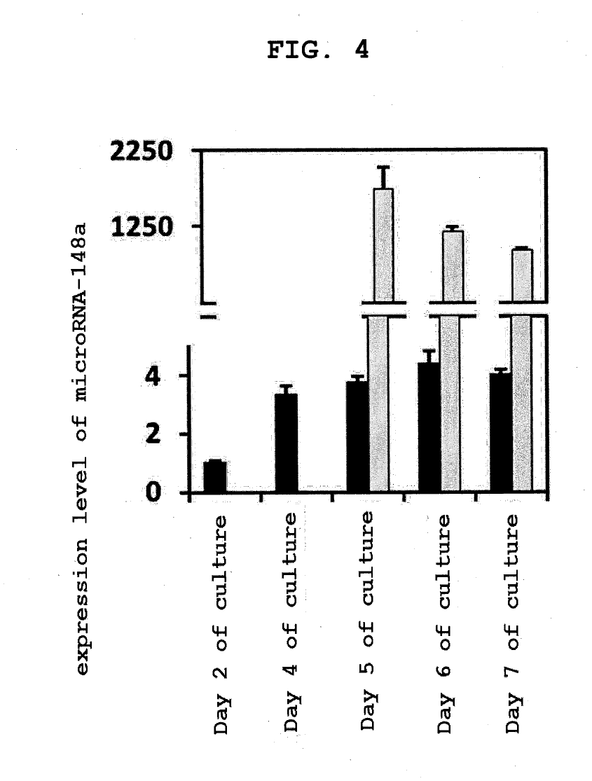

As shown in FIG. 4, miR-148a was overexpressed from day 5 of culture after introduction of miRNA.

### Example 5

### Changes in Expression Level of Hepatocyte Marker after Differentiation Induction

Mouse hepatic progenitor cells were plated at 4×10⁴ cells/cm² in a 35 mm cell culture dish coated with type I collagen, and cultured in a medium for hepatic progenitor cells. miR-148a precursor was introduced into the cultured mouse hepatic progenitor cells and, after confluence (after day 4 of culture), the cells were cultured in the medium for hepatic progenitor cells free of epidermal growth factor (EGF) and fetal bovine serum. Total RNA was extracted, and the expression levels of genes of albumin, glucose-6-phosphatase (G6pc) present in hepatocyte, tyrosine amino group transferase (Tat), drug-metabolizing enzyme (Cyp17a1), which are hepatocyte marker, and miR-122 which is liver-specifically expressed were quantified by the real-time PCR method. When miR-148a precursor was not introduced, the measurement was started from day 2 of culture; when miR-148a precursor was introduced, the measurement was started from day 5 of culture after introduction of miRNA.

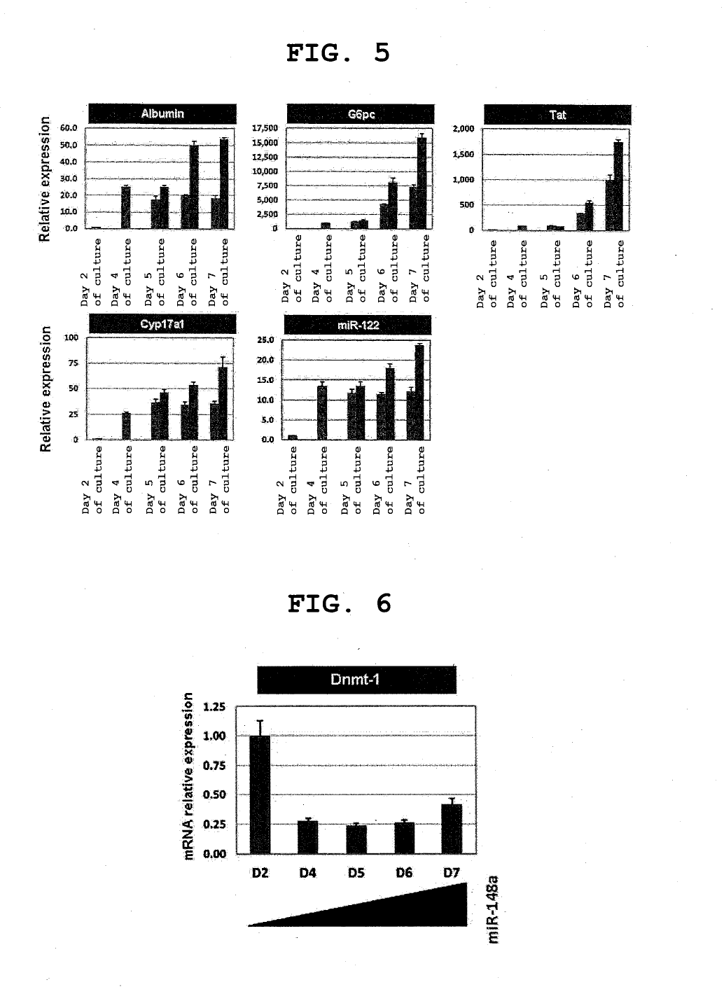

As shown in FIG. 5, the expression levels of albumin, G6pc, Tat, Cyp17a1 and miR-122 all increased after differentiation induction. When miR-148a precursor was introduced, high expression levels were observed from day 5 of culture, as compared to no introduction of miR-148a precursor.

### Example 6

### Changes in Dnmt-1 Expression Level after Differentiation Induction

Mouse hepatic progenitor cells were plated at 4×10⁴ cells/cm² in a 35 mm cell culture dish coated with type I collagen, and cultured in a medium for hepatic progenitor cells. After confluence (after day 4 of culture), the cells were cultured in the medium for hepatic progenitor cells free of epidermal growth factor (EGF) and fetal bovine serum. The medium was exchanged every day. Total RNA was extracted, and Dnmt-1 was quantified by the real-time PCR method.

As shown in FIG. 6, the Dnmt-1 expression level decreased after differentiation induction, contrary to the miR-148 expression level.

### Example 7

### Changes in Dnmt-1 Expression Level after Differentiation Induction of Hepatic Progenitor Cell Introduced with miR-148a Precursor

Mouse hepatic progenitor cells were plated at 4×10⁴ cells/cm² in a 35 mm cell culture dish coated with type I collagen, and cultured in a medium for hepatic progenitor cells. miR-148a precursor was introduced into the cultured mouse hepatic progenitor cells and, after confluence (after day 4 of culture), the cells were cultured in the medium for hepatic progenitor cells free of epidermal growth factor (EGF) and fetal bovine serum. The medium was exchanged every day. Total RNA was extracted, and Dnmt-1 was quantified by the real-time PCR method.

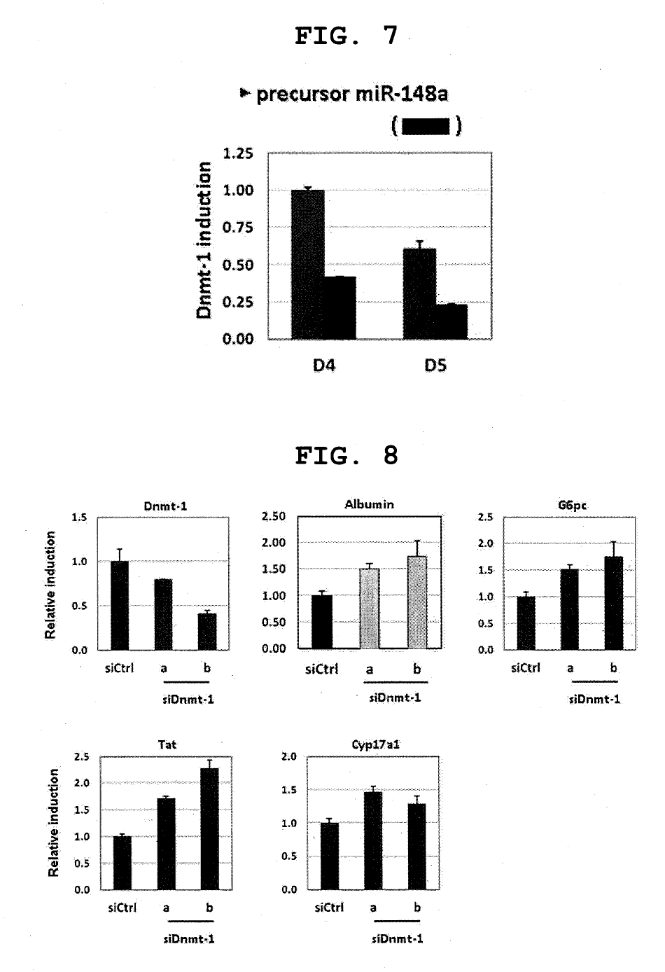

As shown in FIG. 7, the Dnmt-1 expression levels on days 4 and 5 of culture further decreased when miR-148a precursor was introduced, as compared to no introduction of miR-148a precursor. The results show that introduction of miR-148a can suppress the expression of Dnmt-1 to a lower level in differentiation induction into hepatocyte.

### Example 8

### Changes in Expression Level of Hepatocyte Marker after Differentiation Induction, Due to Inhibition of Dnmt-1 Expression

Mouse hepatic progenitor cells were plated at 4×10⁴ cells/cm² in a 35 mm cell culture dish coated with type I collagen, and cultured in a medium for hepatic progenitor cells. siRNA that suppresses expression of Dnmt-1 (siDnmt-la or b mentioned below) was introduced into the cultured mouse hepatic progenitor cells and, after confluence (after day 4 of culture), the cells were cultured in the medium for hepatic progenitor cells free of epidermal growth factor (EGF) and fetal bovine serum. On day 6 of culture, total RNA was extracted. The expression level of Dnmt-1, and the expression level of genes of albumin, glucose-6-phosphatase (G6pc) present in hepatocyte, tyrosine amino group transferase (Tat), and drug-metabolizing enzyme (Cyp17a1), which are hepatocyte marker, were quantified by the real-time PCR method. The expression levels of genes of hepatocyte marker albumin, glucose-6-phosphatase present in hepatocyte and drug-metabolizing enzyme (G6pc, Tat and Cyp) were quantified by the real-time PCR method.

| siDnmt-1a: | |
|---|---|
| (SEQ ID NO: 9) | |
| sense strand: | GGUAGAGAGUUACGACGAAtt |
| (SEQ ID NO: 10) | |
| antisense strand: | UUCGUCGUAACUCUCUACCtg |
| siDnmt-1b: | |
| (SEQ ID NO: 11) | |
| sense strand: | CAACGGAUCCUAUCACACUtt |
| (SEQ ID NO: 12) | |
| antisense strand: | AGUGUGAUAGGAUCCGUUGta |

As shown in FIG. 8, when siDnmt-la or b was introduced, high expression levels were found in either gene as compared to introduction of control siRNA (siCtrl) that does not suppress expression of Dnmt-1. In addition, the expression of Dnmt-1 was suppressed as compared to introduction of control siRNA (siCtr1).

### Example 9

### Changes in Expression Level of miR-148a after Differentiation Induction, Due to Substance that Inhibits DNA Methylation Caused by Dnmt-1

Mouse hepatic progenitor cells were plated at 1×10⁵ cells/cm² in a 35 mm cell culture dish coated with type I collagen, and cultured in a medium for hepatic progenitor cells. On day 2 of culture, the medium was exchanged with a medium for hepatic progenitor cell, containing a substance that inhibits DNA methylation caused by Dnmt-1 (5-aza-2′-deoxycytidine (decitabine): 0.2 μM or 1.0 μM, or RG108: 20 μM or 100 μM), and the mouse hepatic progenitor cells were cultured to confluence. After confluence, the cells were cultured in the medium for hepatic progenitor cells free of epidermal growth factor (EGF) and fetal bovine serum but added with a substance that inhibits DNA methylation (5-aza-2′-deoxycytidine (decitabine): 0.2 μM (5-aza 0.2) or 1.0 μM (5-aza 1.0), or RG108: 20 μM (RG 20) or 100 μM (RG 100)), whereby differentiation was induced. The medium was exchanged every day. On day 7 of culture, total RNA was extracted, and the miR-148a expression level was quantified by the real-time PCR method.

As shown in FIG. 9, the expression of miR-148a showed a high level when differentiation was induced in a medium containing a substance that inhibits DNA methylation caused by Dnmt-1, as compared to differentiation induction in a medium without addition of a substance that inhibits DNA methylation caused by Dnmt-1 (control).

### Example 10

### Changes in Expression Level of Hepatocyte Marker after Differentiation Induction, Due to Substance that Inhibits DNA Methylation Caused by Dnmt-1

Mouse hepatic progenitor cells were plated at 1×10⁵ cells/cm² in a 35 mm cell culture dish coated with type I collagen, and cultured in a medium for hepatic progenitor cells. On day 2 of culture, the medium was exchanged with a medium for hepatic progenitor cell, containing a substance that inhibits DNA methylation caused by Dnmt-1 (5-aza-2′-deoxycytidine (decitabine): 0.2 μM or 1.0 μM, or RG108: 20 μM or 100 μM), and the mouse hepatic progenitor cells were cultured to confluence. After confluence, the cells were cultured in the medium for hepatic progenitor cells free of epidermal growth factor (EGF) and fetal bovine serum but added with a substance that inhibits DNA methylation (5-aza-2′-deoxycytidine (decitabine): 0.2 μM or 1.0 μM, or RG108: 20 μM or 100 μM), whereby differentiation was induced. The medium was exchanged every day. On day 7 of culture, total RNA was extracted, and the expression levels of hepatocyte marker albumin, a drug-metabolizing enzyme (Cyp3a4), and miR-122 that expresses liver-specifically were quantified by the real-time PCR method.

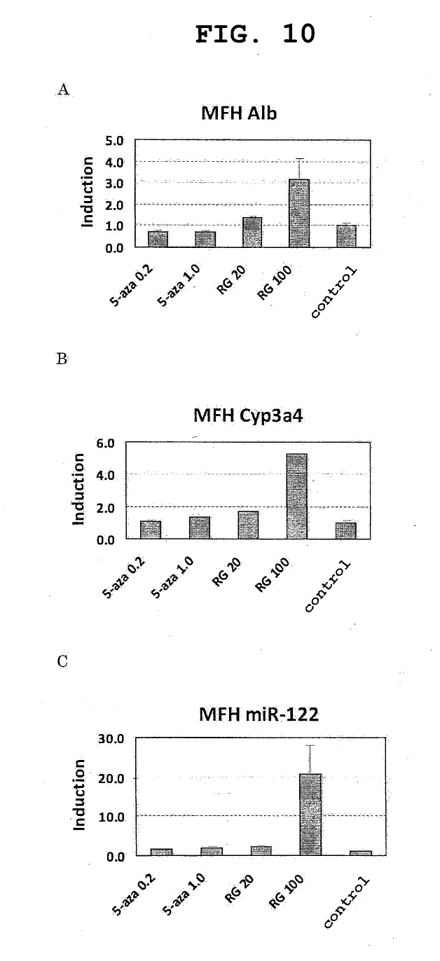

As shown in FIGS. 10B and C, the expression of both Cyp3a4 and miR-122 showed a high level when differentiation was induced in a medium containing a substance that inhibits DNA methylation caused by Dnmt-1, as compared to differentiation induction in a medium without addition of a substance that inhibits DNA methylation caused by Dnmt-1 (control). As shown in FIG. 10A, moreover, the expression of albumin showed a high level when differentiation was induced in a medium containing RG108, as compared to the control. These results show that differentiation into hepatocyte is promoted when differentiation is induced in a medium containing a substance that inhibits DNA methylation caused by Dnmt-1.

### Example 11

### Changes in Expression Level of Hepatocyte Marker after Differentiation Induction in Hepatic Progenitor Cell Introduced with miR-148a Precursor

Human hepatic progenitor cells (Cell System Corporation) were plated at 2×10⁴ cells/cm² on a 6-well plate coated with matrigel (Becton, Dickinson and Company) and cultured. miR-148a precursor was introduced into the cultured human hepatic progenitor cells and, after confluence, the cells were cultured in a medium for hepatic progenitor cell, which contained fetal bovine serum lowered to 1%. The medium was exchanged every day. Total RNA was extracted on days 5, 7 and 9 after miR-148a precursor introduction. The expression levels of the genes of albumin and the drug-metabolizing enzyme (Cyp3A4) which are hepatocyte markers were quantified by the real-time PCR method.

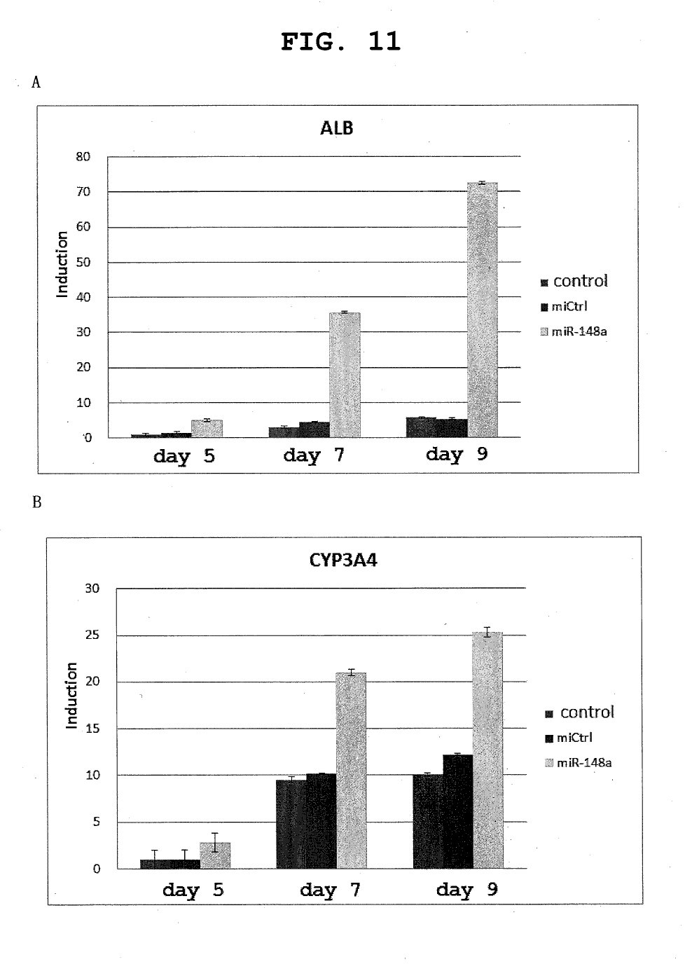

As shown in FIG. 11, the expression levels of albumin and Cyp3A4 showed a high level when miR-148a precursor was introduced, as compared to introduction of miRNA (miCtrl) that does not have function and culture in a medium alone (control).

### Example 12

### Morphology of Hepatic Progenitor Cell Introduced with miR-148a Precursor

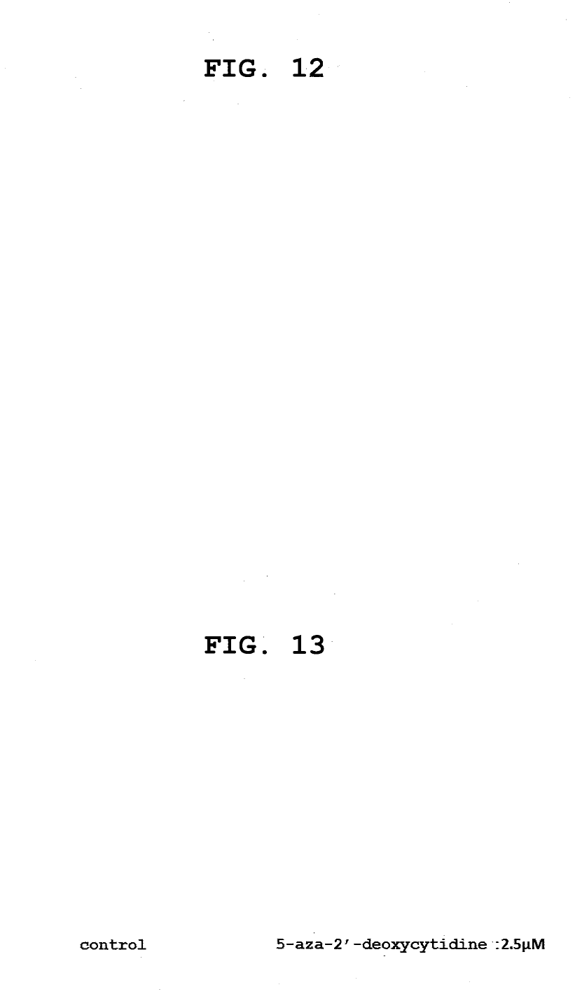

Human hepatic progenitor cells (Cell System Corporation) were plated at 2×10⁴ cells/cm² on a 6-well plate coated with matrigel (Becton, Dickinson and Company) and cultured. miR-148a precursor was introduced into the cultured human hepatic progenitor cells and, after confluence, the cells were cultured in a medium for hepatic progenitor cell, which contained fetal bovine serum lowered to 1%. The medium was exchanged every day. The morphology on day 14 of culture is shown in FIG. 12. A cell having morphology specific to mature hepatocyte (multiple nuclei, clear cell boundary) was observed.

From the above results, it was shown that miR-148a promotes differentiation of hepatic progenitor cell by suppressing expression of Dnmt-1 and achieves differentiation into hepatocyte having high maturity.

### Example 13

### Morphology of HepG2 Cell Cultured in Medium Added with Substance that Inhibits DNA Methylation Caused by Dnmt-1

Hepatoblastoma HepG2 cells (American Type Culture Collection: ATCC) were plated on a 35 mm cell culture dish at 7×10⁵ cells, and cultured in DMEM (Invitrogen Corporation) added with 100 IU/mL penicillin-100 μg/mL streptomycin solution (Invitrogen Corporation), 10% fetal bovine serum (Thermo Fisher Scientific K.K.), and a substance that inhibits DNA methylation (5-aza-2′-deoxycytidine: 5-aza (decitabine): 2.5 μM). The medium was exchanged every day. The morphology on day 14 of culture is shown in FIG. 13. HepG2 cells cultured in a medium added with a DNA methylation inhibitor had morphology specific to mature hepatocyte (multiple nuclei, clear cell boundary).

### Example 14

### PAS Staining of HepG2 Cell Cultured in Medium Added with Substance that Inhibits DNA Methylation Caused by Dnmt-1

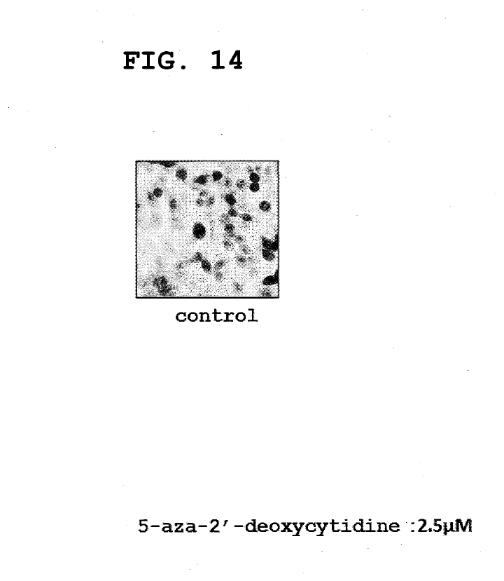

HepG2 cells (ATCC) were plated on a 35 mm cell culture dish at 7×10⁵ cells, and cultured in DMEM (Invitrogen Corporation) added with 100 IU/mL penicillin-100 μg/mL streptomycin solution (Invitrogen Corporation), 10% fetal bovine serum (Thermo Fisher Scientific K.K.), and a substance that inhibits DNA methylation (5-aza-2′-deoxycytidine: 5-aza (decitabine): 2.5 μM). The medium was exchanged every day. After culture for 14 days, PAS staining was performed to confirm storage capacity of glycogen which is found in mature hepatocyte (FIG. 14). The cells added with a DNA methylation inhibitor showed high glycogen storage capacity as compared to control without addition of the inhibitor.

### Example 15

### Changes in Expression Level of Hepatocyte Marker in HepG2, by Substance that Inhibits DNA Methylation by Dnmt-1

Total RNA was extracted from HepG2 cells by using miRNeasy Mini Kit (QUIAGEN). Total RNA was quantified by NanoDrop 1000 spectrophotometer (Thermo Fisher Scientific K.K.).

Total RNA sample was treated with DNase (TURBO DNA-free kit: Ambion Inc), and cDNA was synthesized from 1 μg of total RNA by using reverse transcriptase (SuperScript III Reverse Transcriptase: Ambion Inc). For quantification, SYBR Green (PlatinumSYBR Green quantitative PCR SuperMix UDG: Invitrogen Corporation) was used. The reaction for real-time PCR was performed at 95° C., 2 min, 40 cycles (95° C. for 15 sec, 60° C. for 30 sec). The cDNA expression level was standardized with GAPDH. HepG2 cells were plated on a 35 mm cell culture dish at 4×10⁵ cells, and cultured in DMEM (Invitrogen Corporation) added with 100 IU/mL penicillin-100 μg/mL streptomycin solution (Invitrogen Corporation), 10% fetal bovine serum (Thermo Fisher Scientific K.K.), and a substance that inhibits DNA methylation (5-aza-2′-deoxycytidine: 5-aza (decitabine): 2.5 μM). The medium was exchanged every day. After culture for 10 days, total RNA was extracted by the above-mentioned method, and the expression levels of genes of albumin, glucose-6-phosphatase (G6pc), tyrosine amino group transferase (Tat), and drug-metabolizing enzymes (Cyp17a1, Cyp2C19, CYP3A4, and CYP1A2), which are hepatocyte markers, were quantified by the real-time PCR method.

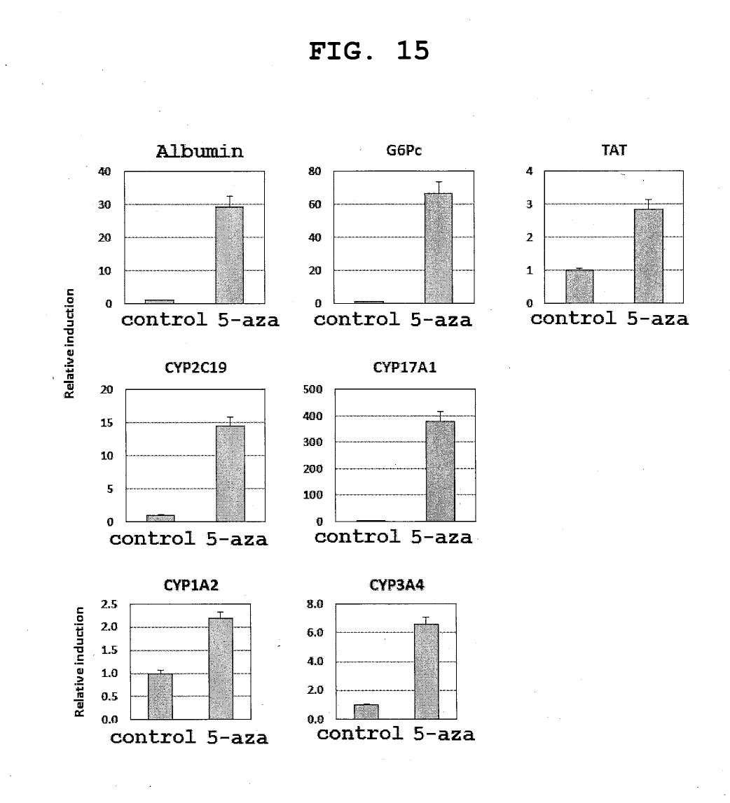

As shown in FIG. 15, the expression of any hepatocyte marker showed a high level when differentiation was induced in a medium containing a DNA methylation inhibitor, as compared to differentiation induction in a medium without addition of the inhibitor (control).

### Example 16

### Changes in CYP Enzyme Activity of Hepatocyte Due to Substance that Inhibits DNA Methylation Caused by Dnmt-1 and Dnmt-1 Inhibitory Nucleic Acid

HepG2 cells were plated on a 12 well plate at 3×10⁵ cells, and cultured in DMEM (Invitrogen Corporation) added with 100 IU/mL penicillin-100 μg/mL streptomycin solution (Invitrogen Corporation), 10% fetal bovine serum (Thermo Fisher Scientific K.K.), and a substance that inhibits DNA methylation (5-aza-2′-deoxycytidine: 5-aza (decitabine): 2.5 μM, 5 μM, 7.5 μM, 10 μM, or 20 μM, or RG108: 20 μM or 100 μM). The medium was exchanged every day. On day 13 of culture, CYP activity was evaluated using P450-Glo CYP450 Assay System (Promega). As for siRNA (siDnmt-1) or miR-148a precursor that suppresses expression of Dnmt-1, CYP activity was evaluated by adding 20 nM of siRNA or miR-148a precursor, and 5 μL of a transfectin reagent (Bio-Rad) to 1 mL of OptiMEM (Invitrogen Corporation) after 72 hr of culture.

After CYP activity measurement, the medium was exchanged with DMEM (Invitrogen Corporation) added with 100 IU/mL penicillin-100 μg/mL streptomycin solution (Invitrogen Corporation), and 10% fetal bovine serum (Thermo Fisher Scientific K.K.), and Cell Counting Kit-8 (DOJINDO LABORATORIES) was added. The absorbance (450 nm) was measured 2 hr later, and the CYP activity was normalized with the cell number.

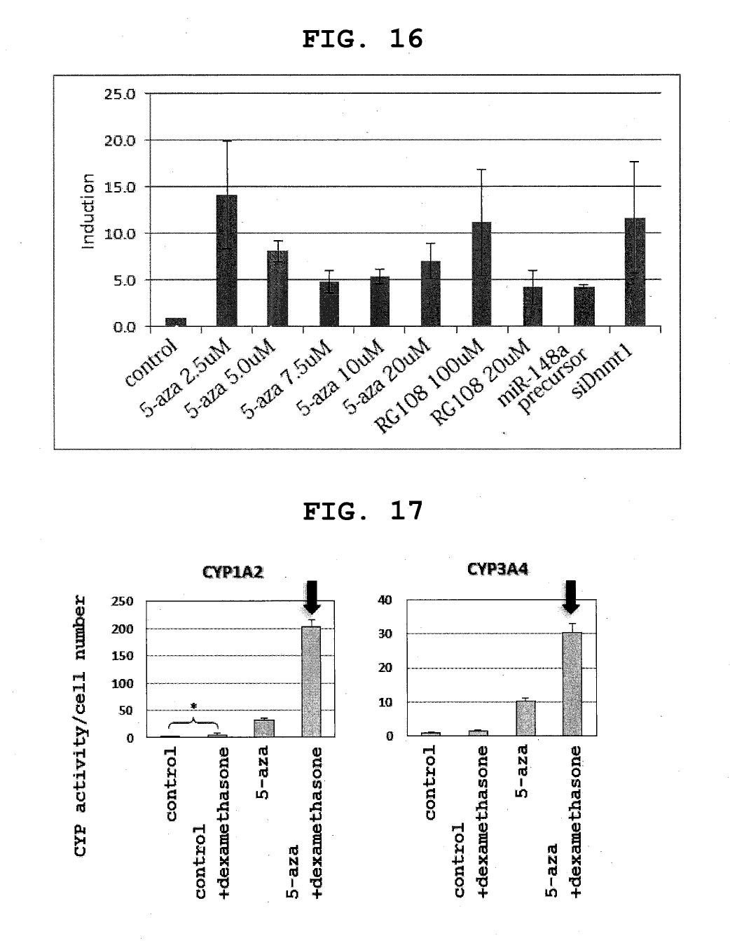

The cells treated with a substance that inhibits DNA methylation caused by Dnmt-1 or Dnmt-1 inhibitory nucleic acid showed high CYP enzyme activity as compared to cells without the treatment (control) (FIG. 16).

### Example 17

### CYP Enzyme Induction Capacity of Hepatocyte Treated with Substance that Inhibits DNA Methylation Caused by Dnmt-1

HepG2 cells were plated on a 35 mm cell culture dish at 1×10⁶ cells, and cultured in DMEM (Invitrogen Corporation) added with 100 IU/mL penicillin-100 μg/mL streptomycin solution (Invitrogen Corporation), 10% fetal bovine serum (Thermo Fisher Scientific K.K.), and a substance that inhibits DNA methylation (5-aza-2′-deoxycytidine: 5-aza (decitabine): 2.5 μM). The medium was exchanged every day. Dexamethasone (25 μM) was added after 1 week of culture. After 72 hr of culture, CYP activity induction capacity was evaluated using P450-Glo CYP450 Assay System (Promega) (FIG. 17).

When enzyme induction was performed by adding dexamethasone to hepatocyte treated with a medium containing a substance that inhibits DNA methylation caused by Dnmt-1, high enzyme induction capacity of CYP1A2 and CYP3A4 was observed as compared to differentiation induction in a medium without addition of the inhibitor (control).

From the above results, it was shown that hepatoblastoma treated with a substance that inhibits DNA methylation caused by Dnmt-1 or Dnmt-1 inhibitory nucleic acid differentiates into hepatocyte with higher maturity.

### INDUSTRIAL APPLICABILITY

According to the present invention, hepatocyte (preferably hepatocyte with high maturity) can be provided in a short period, which enables stable supply of hepatocytes usable for experiments for the prediction of hepatotoxicity and liver metabolite and the like. Using hepatic progenitor cell induced from stem cell having superior proliferation capacity in the method of the present invention, hepatocytes with less lot-to-lot variation than primary cultured hepatocytes can be produced stably. Therefore, hepatocytes suitable for continuous use in drug screening and the like can be provided.

This application is based on Japanese patent application Nos. 2012-177171 (filing date: Aug. 9, 2012) and 2013-003365 (filing date: Jan. 11, 2013), the contents of which are encompassed in full herein.

1. A promoter of differentiation from hepatic progenitor cell or hepatoblastoma into hepatocyte, which comprises a substance that suppresses expression of Dnmt-1 or a substance that inhibits function of Dnmt-1 as an active ingredient.

2. The differentiation promoter according to claim 1, wherein the substance that suppresses expression of Dnmt-1 is a nucleic acid that suppresses expression of Dnmt-1 or a precursor thereof.

3. The differentiation promoter according to claim 2, wherein the nucleic acid that suppresses expression of Dnmt-1 or precursor thereof is miRNA for mRNA of Dnmt-1 gene or a precursor thereof, or siRNA for mRNA of Dnmt-1 gene or a precursor thereof.

4. The differentiation promoter according to claim 3, wherein the nucleic acid that suppresses expression of Dnmt-1 or precursor thereof is miR-148a or a precursor thereof.

5. The differentiation promoter according to claim 1, wherein the substance that inhibits function of Dnmt-1 is a substance that inhibits DNA methylation caused by Dnmt-1.

6. The differentiation promoter according to claim 1, wherein the substance that inhibits DNA methylation caused by Dnmt-1 is a cytidine analog or a salt, solvate, hydrate, precursor or derivative thereof.

7. The differentiation promoter according to claim 5, wherein the substance that inhibits DNA methylation caused by Dnmt-1 masks a Dnmt-1 target sequence.

8. The differentiation promoter according to claim 5, wherein the substance that inhibits DNA methylation caused by Dnmt-1 blocks an active site of Dnmt-1.

9. The differentiation promoter according to claim 1, wherein the hepatic progenitor cell is a primary cultured cell or a cell induced to differentiate from a stem cell.

10. The differentiation promoter according to claim 9, wherein the hepatic progenitor cell is a primary cultured cell.

11. The differentiation promoter according to claim 9, wherein the hepatic progenitor cell is a cell induced to differentiate from an embryonic stem cell, an artificial pluripotent stem cell or a mesenchymal stem cell.

12. A method of producing a hepatocyte from a hepatic progenitor cell or hepatoblastoma, comprising the following steps:
(1) a step of suppressing expression of Dnmt-1 or inhibiting function of Dnmt-1 in a hepatic progenitor cell or hepatoblastoma by contacting the hepatic progenitor cell or hepatoblastoma with the differentiation promoter according to claim 1; and
(2) a step of inducing differentiation into a hepatocyte by culturing the hepatic progenitor cell or hepatoblastoma obtained in said step (1), wherein expression of Dnmt-1 is suppressed or function of Dnmt-1 is inhibited.

13. The method according to claim 12, wherein the hepatic progenitor cell is a primary cultured cell a cell induced to differentiate from or a stem cell.

14. The method according to claim 13, wherein the hepatic progenitor cell is a primary cultured cell.

15. The method according to claim 13, wherein the hepatic progenitor cell is a cell induced to differentiate from an embryonic stem cell, an artificial pluripotent stem cell or a mesenchymal stem cell.

16. A hepatocyte produced by the method according to claim 12.

17. A method of evaluating metabolism of a test compound, comprising the steps of the following (a) and (b):
(a) a step of contacting the hepatocyte according to claim 16 with a test compound; and
(b) a step of measuring the metabolism of the test compound contacted with the hepatocyte in said step (a).

18. A method of evaluating hepatotoxicity of a test compound, comprising the steps of the following (a) and (b):
(a) a step of contacting the hepatocyte according to claim 16 with a test compound; and
(b) a step of measuring the level of disorder of the hepatocyte contacted with the test compound in said step (a).

19. (canceled)

20. The method according to claim 12, wherein the hepatic progenitor cell or hepatoblastoma obtained in step (1), wherein expression of Dnmt-1 is suppressed or function of Dnmt-1 is inhibited, is cultured for not less than 7 days.
